(19) United States
(12) Patent Application Publication
(10) Pub. No.: US 2023/0225838 A1
(43) Pub. Date: Jul. 20, 2023

(54) Cotton Gauze Replacement for Temporary Use in an Oral Cavity

(71) Applicant: Queventive, LLC, Palm Beach, FL (US)

(72) Inventor: Joseph Quevedo, Rochester, NY (US)

(21) Appl. No.: 18/157,069

(22) Filed: Jan. 19, 2023

Related U.S. Application Data
(63) Continuation-in-part of application No. 17/913,019, filed on Sep. 20, 2022.
Application No. 17/913,019 is a continuation-in-part of application No. PCT/US21/21258, filed on Mar. 5, 2021.
(60) Provisional application No. 63/300,881, filed on Jan. 19, 2022.
(60) Provisional application No. 62/992,177, filed on Mar. 20, 2020.
(60) Provisional application No. 62/992,177, filed on Mar. 20, 2020.
(60) Provisional application No. 62/985,731, filed on Mar. 5, 2020.

(51) Int. Cl. A61C 8/00

(52) U.S. Cl. CPC A61C 8/0089; A61C 8/0083; A61C 2201/00

(57) ABSTRACT

A dental surgical article is manufactured by combining one or more isocyanates with one or more polyols including at least one polyol that has a molecular weight of at least 2000.

### CROSS-REFERENCE TO RELATED APPLICATIONS

This patent application claims the benefit of priority to U.S. provisional patent application No. 63/300,881, filed Jan. 19, 2022.

This patent application is a continuation in part of U.S. patent application Ser. No. 17/913,019, filed Sep. 20, 2022, which is a 371 of PCT/US21/23544, filed Mar. 22, 2021, which claims the benefit of priority to U.S. provisional patent application Ser. No. 62/992,177, filed Mar. 20, 2020, and which is a continuation in part of PCT/US21/21258, filed Mar. 5, 2021, which claims the benefit of priority to U.S. provisional patent applications Nos. 62/992,177, filed Mar. 20, 2020 and 62/985,731, filed Mar. 5, 2020.

All of the above priority patent applications are incorporated by reference.

### BACKGROUND OF THE INVENTION

Dental implant surgeries and endodontal repair surgeries for, respectively, replacing and repairing a decaying tooth, a partially or entirely missing tooth, or an otherwise painful, unsightly, or unsuitable tooth are dental surgical options provided by dentists for resolving periodontal issues for their patients. Cotton and gauze have been used as spacer materials to preserve the way for subsequently coupling abutments and crowns to installed implants following osseointegration and/or for facilitating reaccess to a coupling component or to a cavity or canal pathway in a follow-on checkup or procedure. However, cotton and gauze can become sticky and disheveled over time, especially when soaked with bodily fluids, and straggling cotton fibers can provide pathways for microbes. It is desired to have alternative spacer articles and materials that exhibit sufficient porosity and autoclavability and can maintain their structural integrity, even when soaked in bodily fluids and subjected to oral vicissitudes, over extended periods of time.

Gingival retraction involves deflection of marginal gingiva away from a tooth. There exist multiple varieties of mechanical, chemo-mechanical, cordless and surgical retraction techniques. Retraction cords, chemical reagents, electrosurgery, laser tissue sculpting and hemostatic materials are often used when atraumatic displacement of gingival tissue is desired. Of these, gingival retraction cords are most commonly used, often in combination with chemical solutions, astringent gels, or hemostatic agents such as aluminum chloride which can cause gingival recession and can damage epithelial tissue and underlying connective tissues.

Gingival electrosurgery may be used for crevicular troughing but at a significant risk of causing long-term damage.

Retraction pastes have advantages such as comfort reported by patients, faster techniques, ease of use, no need for anesthesia, and reduced tissue trauma. Retraction pastes tend to perform less effectively at the deeper subgingival sites of deeper implants. Injectable materials can be used to form an expanding matrix to provide gingival retraction. As with retraction pastes, injectable matrices provide less effective retraction performance in procedures involving deeper implants.

### BRIEF DESCRIPTION OF DRAWINGS

FIG. 1A illustrates a qube having a size, shape and color that has been selected in accordance with a specific use and function during an oral surgery in accordance with an example embodiment.

FIG. 1B illustrates three qubes in accordance with example embodiments having different sizes and shapes selected, and that may have been optionally cut from a larger qube, such as that illustrated at FIG. 1A.

FIGS. 2A-2G illustrate examples of qubes for use in various roles during oral surgical procedures in accordance with example embodiments.

FIG. 3 illustrated a wedge that is an example of a tapered shape of a dental implant qube or endoqube in accordance with example embodiments.

FIGS. 4A-4F illustrate seven types of qubes in accordance with example embodiments.

FIGS. 5A-5G illustrate schematically examples of retraction qube placement and removal tools having smooth, blunt appendages for placement of a qube for retraction, or for spacing, cushioning, bandaging, or protecting gum tissue around a bone graft site, tooth extraction site or other oral surgical site in accordance with example embodiments.

FIGS. 6A-6F illustrate schematically examples of implant qube and endoQube placement and removal tools in accordance with example embodiments.

FIGS. 7A-7G illustrate qube placement and removal tools in accordance with example embodiments.

FIGS. 8A-8I illustrate photographically certain steps in a process leading incrementally to completion of the coupling of a dental implant at a site of tooth extraction, tooth absence, tooth loss or tooth decay.

FIG. 8A includes a photograph that illustrates an incision site reflected in a mirror in accordance with an example embodiment.

FIG. 8B includes a photograph of a retraction qube inserted into an underside of a surgical flap for retraction in accordance with an example embodiment.

FIG. 8C includes a photograph of a qube further inserted under a flap in accordance with an example embodiment.

FIG. 8D includes a photograph including qubes placed on both sides of an incision to retract a surgical flap and expose underlying bone in accordance with an example embodiment.

FIG. 8E includes a photograph of a visible qube retracting a flap in accordance with an example embodiment.

FIG. 8F includes a photograph that illustrates a retraction qube allowing access to osteotomy for implant site preparation and better visibility in accordance with an example embodiment.

FIG. 8G includes a photograph wherein the retraction qube of FIG. 8F has been removed in accordance with an example embodiment.

FIG. 8H includes a photograph of a qube removed from an opposite side of an implant in place below a gingival surface in accordance with an example embodiment.

FIG. 8I includes a photograph of a surgical flap closed and sutured around a dental implant site in accordance with an example embodiment.

FIGS. 9A-9U illustrate photographically examples of endocubes and/or dental implant cubes in an example endodontal oral surgical repair procedure in accordance with example embodiments.

FIGS. 10A-10I include surgical photographs that include example embodiments of retraction qubes as demonstrably suitable retraction media.

FIGS. 11A-11K illustrate example embodiments of example retraction steps and example retraction qubes useful in multiple roles during a dental implant procedure involving a screw-coupled dental implant in accordance with example embodiments.

FIGS. 12A-12J illustrate example embodiments of example retraction steps and example retraction qubes useful in multiple roles during a dental implant procedure involving a cement-coupled dental implant in accordance with example embodiments.

FIGS. 13A-13G photographically illustrate further example qube uses and applications and example qubes.

FIG. 14A-14T illustrate photographically an example procedure using retraction qubes in accordance with another embodiment.

FIGS. 15A-15K schematically illustrate example embodiments of grafting processes for preparing a bone socket site for coupling a dental implant thereto.

FIGS. 16A-16C schematically illustrate a 3 mm thick, square foam qube in perspective, front and side views, respectively, in accordance with an example embodiment.

FIGS. 17A-17C schematically illustrate a 2 mm thick, square foam qube in perspective, front and side views, respectively, in accordance with an example embodiment.

FIGS. 18A-18C schematically illustrate a 10 mm thick, U-shaped foam qube, in perspective, front and side views, respectively, in accordance with an example embodiment.

FIGS. 19A-19C schematically illustrate a 12 mm thick, U-shaped foam qube, in perspective, front and side views, respectively, in accordance with an example embodiment.

FIGS. 20A-20C schematically illustrate a 15 mm thick, U-shaped foam qube, in perspective, front and side views, respectively, in accordance with an example embodiment.

FIGS. 21A-21C schematically illustrate an 8 mm thick, short rectangular foam qube, in perspective, front and side views, respectively, in accordance with an example embodiment.

FIGS. 22A-22C schematically illustrate a 10 mm thick, short rectangular foam qube, in perspective, front and side views, respectively, in accordance with an example embodiment.

FIGS. 23A-23C schematically illustrate an 8 mm thick, 2D foam mushroom qube, in perspective, front and side views, respectively, in accordance with an example embodiment.

FIGS. 24A-24C schematically illustrate a 10 mm thick, 2D foam mushroom qube, in perspective, front and side views, respectively, in accordance with an example embodiment.

FIGS. 25A-25C schematically illustrate a 10 mm thick, 2D foam football qube, in perspective, front and side views, respectively, in accordance with an example embodiment.

FIGS. 26A-26C schematically illustrate an 8 mm thick, 2D foam thimble qube, in perspective, front and side views, respectively, in accordance with an example embodiment.

FIGS. 27A-27C schematically illustrate a 10 mm thick, 2D foam thimble qube, in perspective, front and side views, respectively, in accordance with an example embodiment.

FIGS. 28A-28C schematically illustrate a 6 mm thick, long rectangular foam qube, in perspective, front and side views, respectively, in accordance with an example embodiment.

FIGS. 29A-29C schematically illustrate a 4 mm thick, long rectangular foam qube, in perspective, front and side views, respectively, in accordance with an example embodiment.

### DETAILED DESCRIPTION OF THE INVENTION

A dental surgical retraction article is provided that may include a polymeric foam sponge that may be autoclavable at 250° F. and may have a porosity that is not less than a porosity of polyurethane. The dental article may be configured in size and shape for retracting a gingival flap during an oral surgery.

The polymeric foam sponge may include polyurethane foam, such as polyester polyurethane foam or polyether polyurethane foam. The polyurethane foam may exhibit a pore density between 45 ppi and 105 ppi.

The polymeric foam sponge may exhibit an elongated shape. The polymeric foam sponge may include a cylindrical, ellipsoidal, tubular, wedge, prism, ovoid, triovoid, egg or pear shape, or combinations thereof.

The polymeric foam sponge may include polyurethane, polytetrafluoroethylene (PTFE), polyolefin, polyamide-imide, polymethylpentene (PMP), polyoxymethylene (POM), polyaryletherketone (PAEK), polyetheretherketone (PEEK), partially reticulated polyether type polyurethane, polyethyl polyurethane, thermoplastic foam, reactive resin foam, polyurethane foam, reaction injection molding plastic foam, flexible foam, thermoplastic polyurethane, mica-particulated polyurethane, resin-particulated polyurethane, resin-blended polyurethane, porous polyurethane, polyester polyurethane, polyether polyurethane, or polyurethane blend, or combinations thereof.

The polymeric foam sponge may include polyurethane blended with one or more additives for enhancing one or more characteristic material attributes. The one or more additives may include silicon oil, silicone surfactant, polyester, polyether, polyethyl, or molybdenum.

The one or more additives may include ethylene glycol, 1,4-butanediol (1,4-BDO or BDO), 1,6-hexanediol, cyclohexane dimethanol or hydroquinone bis(2-hydroxyethyl) ether (HQEE), or combinations thereof.

The one or more additives may include one or more difunctional, trifunctional or tetrafunctional Hydroxyl compounds or one or more difunctional amine compounds, or combinations thereof.

The one or more additives may include one or more difunctional hydroxyl compounds including Ethylene glycol, Diethylene glycol, Triethylene glycol, Tetraethylene glycol, Propylene glycol, Dipropylene glycol, Tripropylene glycol, 1,3-Propanediol, 1,3-Butanediol, 1,4-Butanediol, Neopentyl glycol, 1,6-Hexanediol, 1,4-Cyclohexanedimethanol, HQEE, Ethanolamine, Diethanolamine, Methyldiethanolamine, or Phenyldiethanolamine, or combinations thereof.

The one or more additives may include one or more trifunctional hydroxyl compounds including Glycerol, Trimethylolpropane, 1,2,6-Hexanetriol, or Triethanolamine, or combinations thereof.

The one or more additives may include one or more tetrafunctional hydroxyl compounds including Pentaerythritol, N,N,N′,N′-Tetrakis, (2-hydroxypropyl), or ethylenediamine, or combinations thereof.

The one or more additives may include one or more difunctional amine compounds including Diethyltoluenediamine or Dimethylthiotoluenediamine, or both.

A dental surgical retraction article is also provided that includes a sustainable green polyhydroxurethane foam sponge formed by combining polyamines and cyclic carbonates with polyols prepared from vegetable oils, dimer fatty acids, or fatty acids, or combinations thereof.

A method of manufacturing a dental surgical retraction article is also provided. The method may involve combining one or more aliphatic or cycloaliphatic isocyanates with one or more polyols including at least one polyether or polyester polyol that has a molecular weight of at least 2000.

The one or more polyols may include polycarbonate, polycaprolactone, polybutadiene, polysulfide, castor oil, soybean oil, cotton seed oil, neem seed oil, vegetable oil, dipropylene glycol, glycerine, or a sorbitol/water solution, or combinations thereof.

The method may also include chemically grafting dispersed styrene-acrylonitrile, acrylonitrile, or polyurea (PHD) polymer solids to a polyether or polyester backbone.

The one or more isocyanates may include 1,6-hexamethylene diisocyanate (HDI), 1-isocyanato-3-isocyanatomethyl-3,5,5-trimethyl-cyclohexane, isophorone diisocyanate (IPDI), or 4,4-diisocyanato dicyclohexylmethane (H12MDI or hydrogenated MDI), or combinations thereof.

A dental implant surgical spacer article is also provided. This dental article may include a polymeric foam sponge that is autoclavable at 250° F. and has a porosity not less than a porosity of polyurethane. The dental article may be configured in size and shape to preserve a volume above a dental implant for coupling an abutment to the dental implant during an osseointegration period.

The polymeric sponge may include a base end opposite a tapered end.

The polymeric sponge may include a tapered end to base end weight density ratio of at least 2:1.

The polymeric sponge may exhibit a conic or truncated conic shape.

The polymeric sponge may include a pyramid or truncated pyramid shape.

An endodontic spacer article may include a polymeric foam sponge that may be configured to temporarily preserve a prepared tooth cavity volume until filling material is ready for filling the cavity volume with permanent filling material.

The polymeric sponge may include an absorbed, adhered or trapped medicinal dosage, or combinations thereof.

The polymeric sponge may include a base end opposite a tapered end.

The polymeric sponge may include a tapered end to base end weight density ratio of at least 2:1.

The polymeric sponge may include a conic or truncated conic shape.

The polymeric sponge may exhibit a pyramid or truncated pyramid shape.

A polymeric foam sponge may be configured to protect sensitive or vulnerable mouth tissue from surgical equipment and ambient exposure during an oral surgery.

A dental surgical retraction method is also provided that may include placing a polymeric foam sponge at a gingival incision location to retract the gingival flap during an oral surgery.

A dental surgical spacer method is also provided. The method may include placing a polymeric foam sponge in a space next to an embedded dental implant to preserve a spacing for coupling an abutment to the dental implant after an osseointegration period.

A dental surgical protection method is also provided. The method may include placing a polymeric foam sponge against sensitive or vulnerable mouth tissue as protection from surgical equipment impacts and ambient exposure during an oral surgery.

A dental article is also provided. The dental article may include a polymeric foam sponge that is autoclavable at 250° F. and has a porosity not less than a porosity of polyurethane and is configured for insertion into a bone socket recess to nonadhesively contact and compress loose graft material contained therein.

The polymeric foam sponge may include polyurethane or polyurethane blend.

The polymeric foam sponge may include a porosity not less than a porosity of polyurethane.

The polymeric foam sponge may be further configured for maintaining a volume density integrity of compressed graft material when removing bodily fluids from the bone socket recess by suctioning said fluids through the polymeric foam sponge.

A guided tissue regeneration membrane may be configured to be disposed between graft material and a polymeric foam sponge during compression of the graft material by applying contact pressure nonadhesively to the sponge.

The membrane may be configured to remain over the graft material within the socket graft recess during an osseointegration period.

A dental bone socket grafting method is also provided. A bone socket recess defined within a patient's jawbone is prepared. After the preparing of the bone socket recess, the bone socket recess is filled with loose graft material. The loose graft material may be compressed within the bone socket recess by inserting a nonadhesive polymeric foam sponge into contact with the loose graft material therein and applying pressure to the sponge. The nonadhesive polymeric foam sponge may exhibit autoclavability at 250° F. and may have a porosity which is not less than a porosity of polyurethane.

The preparing of a bone socket recess may involve shape cutting or drilling into a tooth, or through gum tissue, or into some bone tissue, or combinations thereof.

The preparing of a bone socket recess may involve removing one or more of a decayed tooth, decayed tissue, excess tissue, microbial organic material, or inorganic debris, or combinations thereof.

The method may include suctioning fluid from the bone socket recess through the sponge.

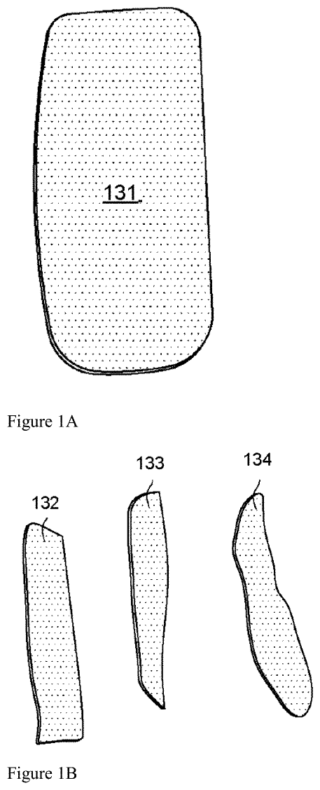

The text descriptions that follow generally refer to specific additional example embodiments that are illustrated schematically and/or in photographs, tables and/or other graphics referred to herein as FIGS. 1A-15K. Many of the components and parameters illustrated as FIGS. 1A-15K embody concepts that are described herein with reference to multiple working examples.

FIG. 1A illustrates a qube having a size, shape and color that has been selected in accordance with a specific use and function during an oral surgery in accordance with an example embodiment.

FIG. 1B illustrates three qubes having different sizes and shapes selected, and optionally cut from, a larger qube, such as that illustrated at FIG. 1A, each for a specific intended use during an oral surgery in accordance with example embodiments. Several qubes of each of several types, shapes, sizes, and compositions are illustrated and described in example embodiments herein.

In some example embodiments, a qube may relate to an article for application to human and animal teeth and human and animal dental implants as a medicated and non-medicated space maintainer and/or retraction medium (referred to herein as a QUBE, a Qube, or a qube). A Qube may include, in an example embodiment, a synthetic sponge-like material with a 1) specific porosity size 2) which is autoclavable 3) which can be colored 4) which can be used a vehicle to carry a medicament 1.2% Chlorohexidine, 5) which can be used a vehicle to carry a medicament Calcium hydroxide Ca(OH), 6) which can be used a vehicle to carry a medicament Povodine-Iodine solution, 7) which can be used a vehicle to carry a medicament 2% Iodine Potassium Iodide, 8) which can be used a vehicle to carry a Sterile saline. The Qube is to be applied as an interappointment dressing for endodontically treated teeth in the access cavity to serve as a barrier from microbial invasion of the canal space as well as a mechanism to prevent damage to surrounding tooth structure when a dentist re-accesses the tooth for permanent restoration. The Qube can also be used as a barrier from microbial invasion within the internal aspect of the coronal access of screw retained dental implants.

The Qube can also be used as a retraction medium for gingival flaps during dental surgery. The Qube can be contoured in specific shapes. The Qube can be impregnated with barium sulfate so it can be visible radiographically. The Qube can be inserted and compacted against gingival soft tissue to allow for atraumatic retraction.

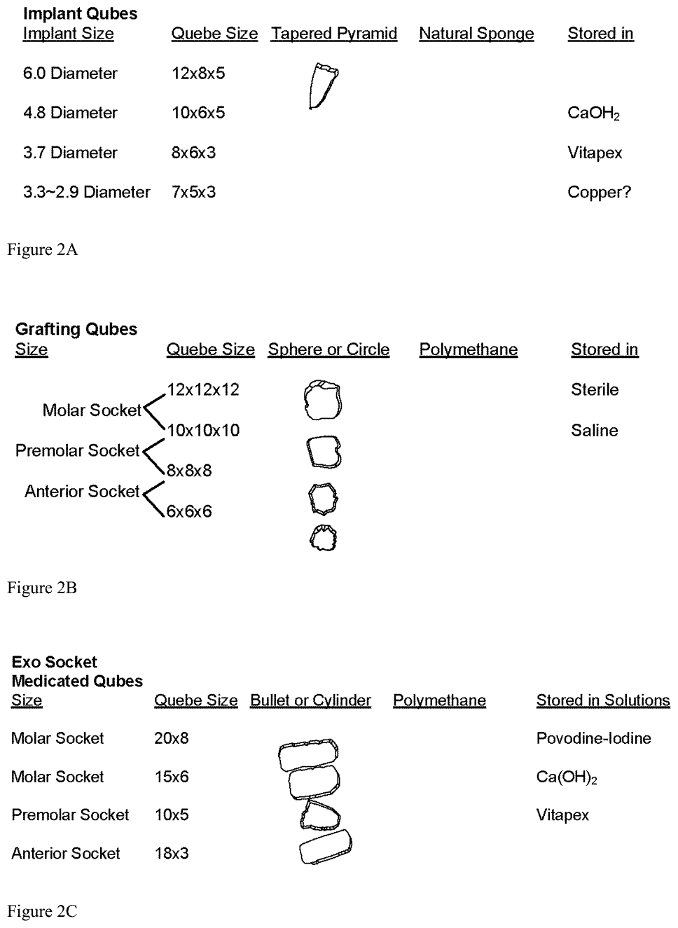

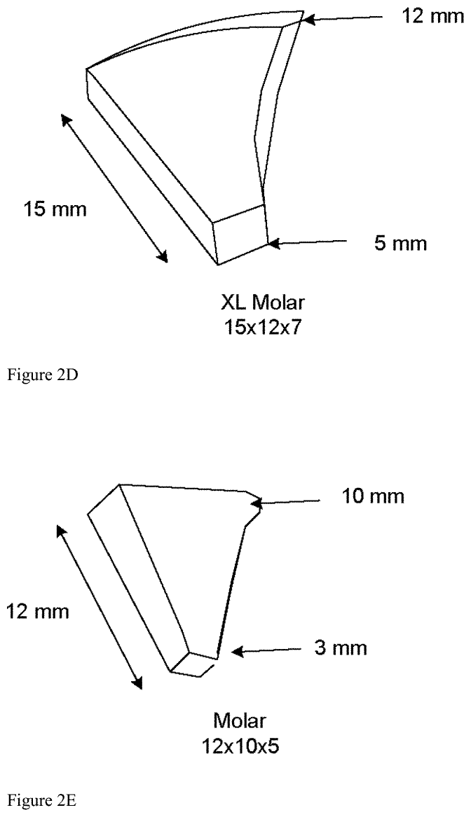

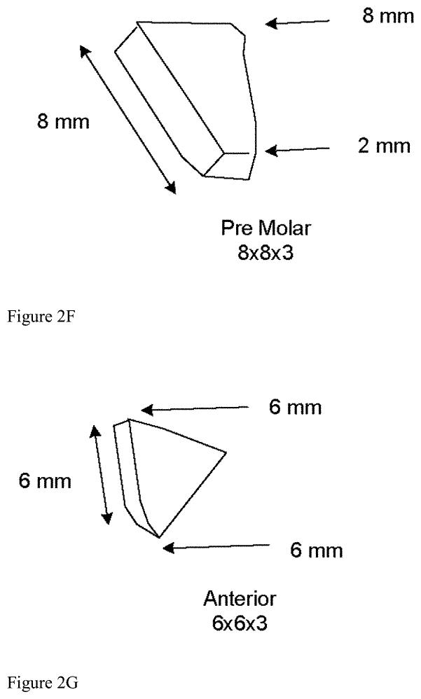

FIGS. 2A-2G illustrate examples of qubes for use in various roles during oral surgical procedures, including as dental implant spacer qubes at FIG. 2A, and as grafting qubes in FIG. 2B, and as exo socket medicated qubes as in FIG. 2C, and as tapered endoQubes in FIGS. 2D-2G.

FIGS. 2A-2C illustrate multiple examples of each of three different qube types, including implant cubes, grafting qubes and endo-socket medicated qubes in accordance with example embodiments.

FIG. 2D-2G illustrates multiple examples of a fourth qube type, including tapered endo qubes in accordance with example embodiments.

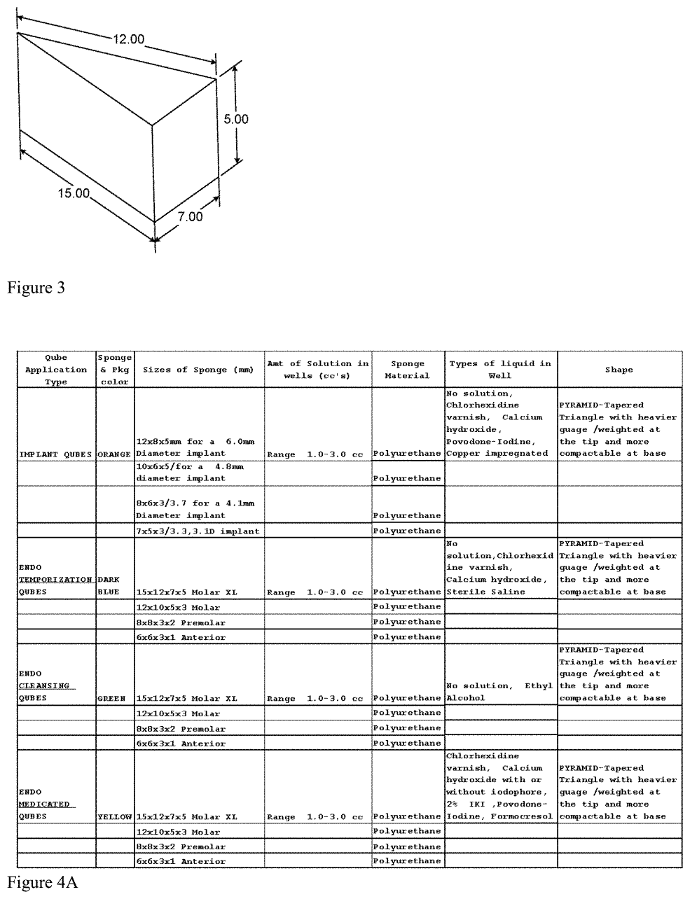

The tapered shape of the wedge shaped qube of FIG. 3 renders it advantageous for insertion into a dental implant space reserved for coupling with an abutment component or an abutment space reserved for coupling with a crown component for a duration of an osseointegration of the implant. The wedge of FIG. 3 has four long sides and a square or rectangular base. Two long sides are parallel, tapered and/or triangularly-shaped and the other two sides are rectangular and form an acute angle at the tapered end.

One or both of the rectangular long sides may also be tapered or be triangularly-shaped, and a pyramidal qube or tetrahedral qube or truncated pyramid or truncated tetrahedron or cone-shaped, four or five sided pyramid, or pentagonal cone, pentagonal pyramid, truncated cone, half ellipsoid or partial ellipsoid or truncated ellipsoid. One or more of the long sides of a regular rectangular box, cube or polyhedron may be tapered or compressed spatially at one end.

An implant qube may be more densely-weighted at a tapered end, which may taper to a point or may round off or may be truncated such that a plane at a tapered end may be parallel to a base plane of greater area of a truncated implant qube, which may have small and large diameter circular end planes, or an elongated end plane quadrilateral having at least one short dimension which may taper to a point in one or both cross-plane dimensions.

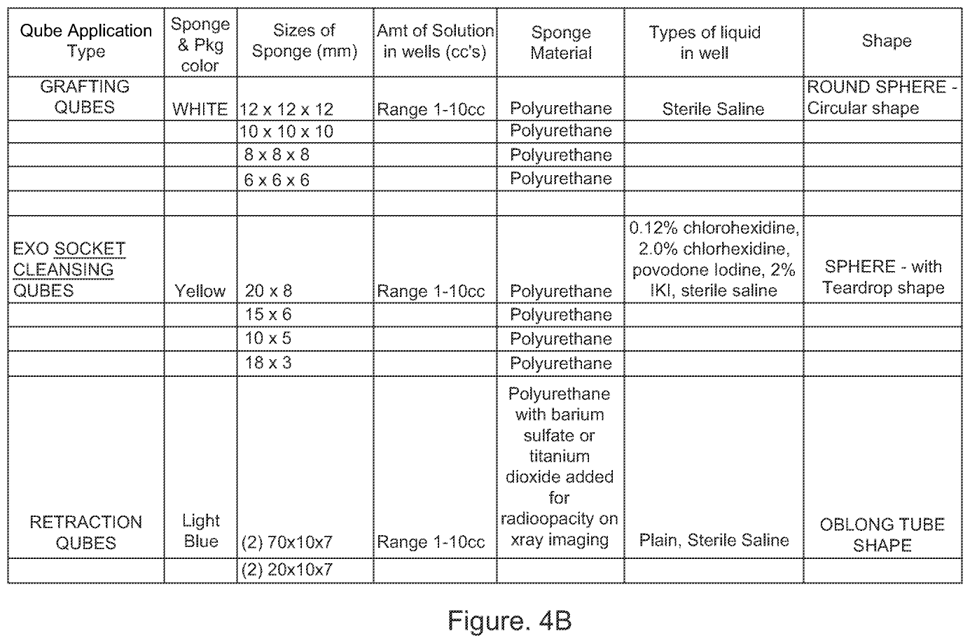

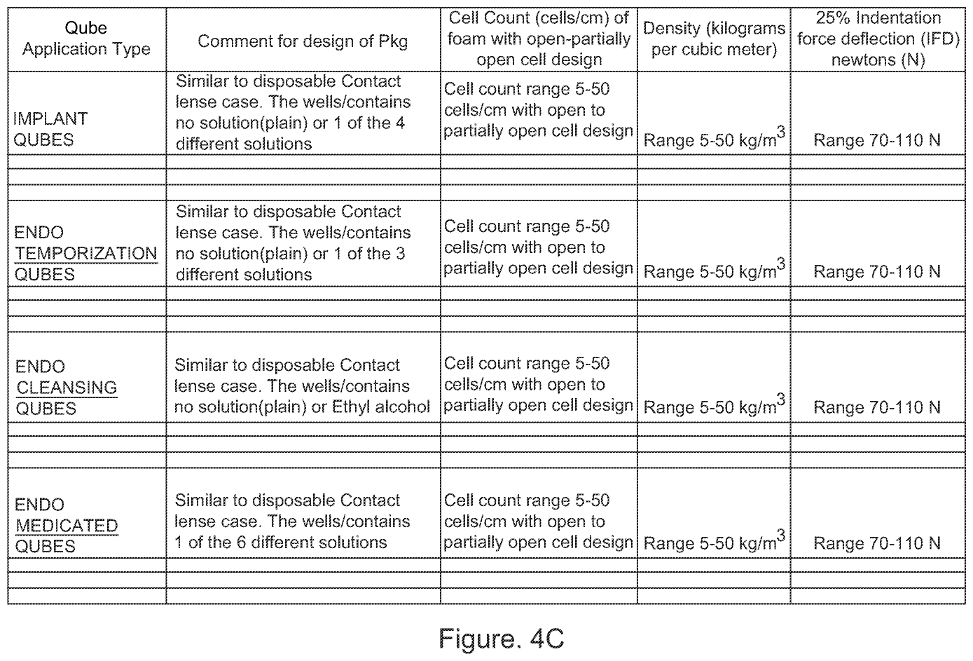

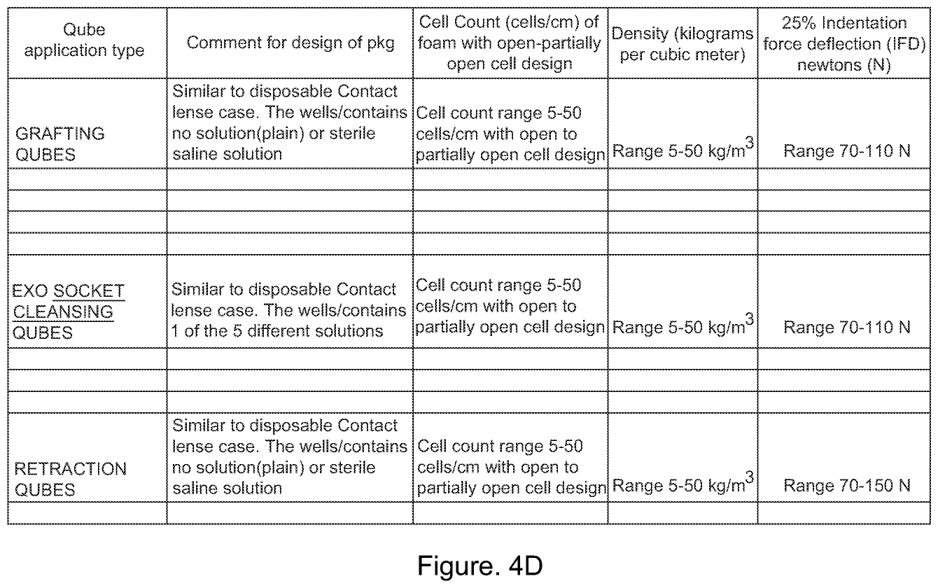

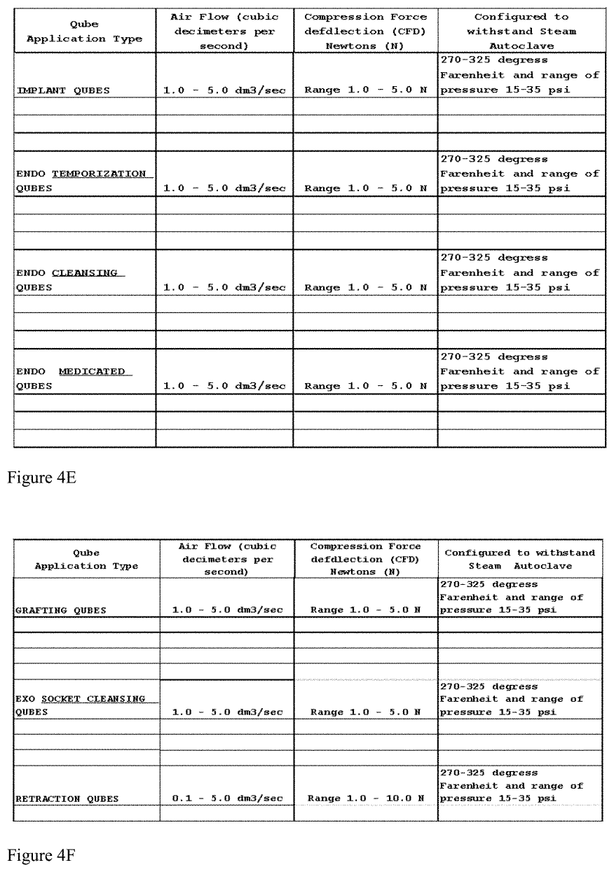

FIGS. 4A-4F illustrate seven types of qubes in accordance with example embodiments. Different qubes may have different physical, chemical or biological properties, different functions, different uses, different roles to play within oral surgical applications, different compositions (polymer units or polymer side chains, molecular component monomers or side chains, monomer units or monomer side groups, different sizes (millimeters to centimeters) and shapes (spheres, ellipsoids, cubes, polyhedrons with four to twenty-four sides, wedges, pyramids, tetrahedrons, tapered polyhedrons, truncated polyhedrons, ovoid) and being grouped according to anticipated, intended or scheduled uses, functions, or specific applications among multiple example oral surgical applications in accordance with example embodiments.

Other qube types may include qubes having different colors or color distributions or different weights, or different overall weight densities (10 kg/m3, 15 kg/m3, 20 kg/m3, 25 kg/m3, 30 kg/m3, 35 kg/m3, 40 kg/m3), or different weight or weight density distributions (20 & 30 kg/m3, or different porosities, autoclavabilities (thermal: 250 F-300 F, 225 F-325 F, 200 F-275 F, 200 F-280 F, 270 F-280 F, 275 F-300 F, 275 F-325 F; pressure: 20 psi-30 psi, 25-35 psi, 25-30 psi, 23-28 psi, 24=27 psi, 28-36 psi, 24-38 psi)-indentation force deflection (IFD) capabilities, cell openness, cell densities (15-16 cells/cm, 10-20 cells/cm, medicament chemistry (calcium hydroxide CaOH, barium sulfide BaS, titanium dioxide TiO2, silver nitride Ag3N, silver nitrate AgNO3, silver ion Ag+, silver ion Ag−, 1.2% chlorohexidine, povodine-iodine 2% iodine potassium iodide, sterile saline), or medicament biology (bacteriocidal, medicament combinational process types (e.g., soaking, coating, encapsulating, embedding, integrating, release rate).

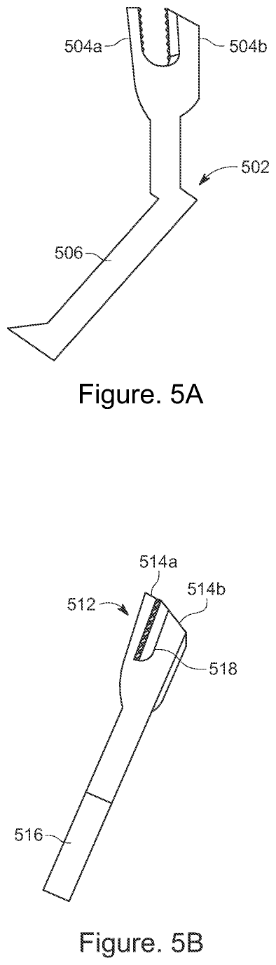

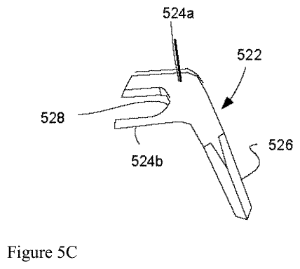

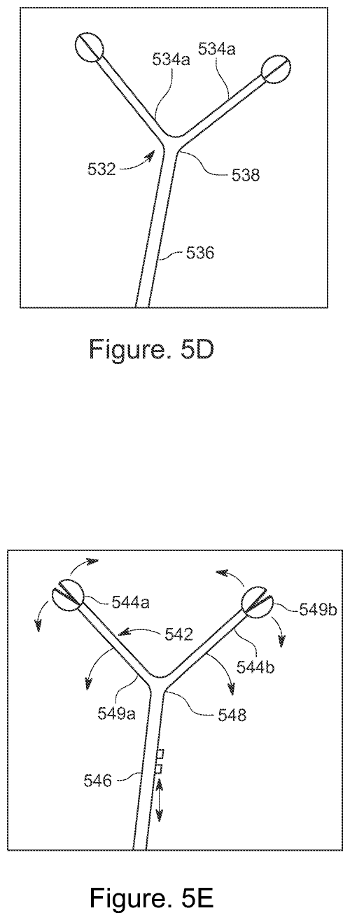

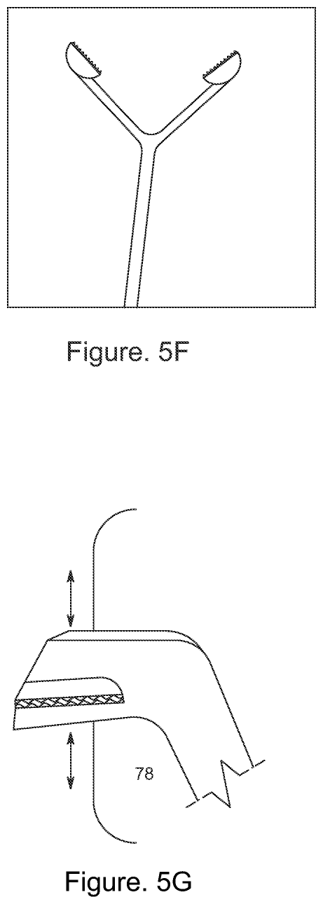

FIGS. 5A-5G illustrates a qube placement tool having smooth, blunt appendages for placement of a qube for retraction, or for spacing, cushioning, bandaging, or protecting gum tissue around a bone graft site, tooth extraction site or other oral surgical site in accordance with an embodiment. The blunt appendages of the qube placement tool may be fixed or may be movable towards or away from each other at one end. Examples include plyers without sharp edges or forks with two or more prongs having rounded ends.

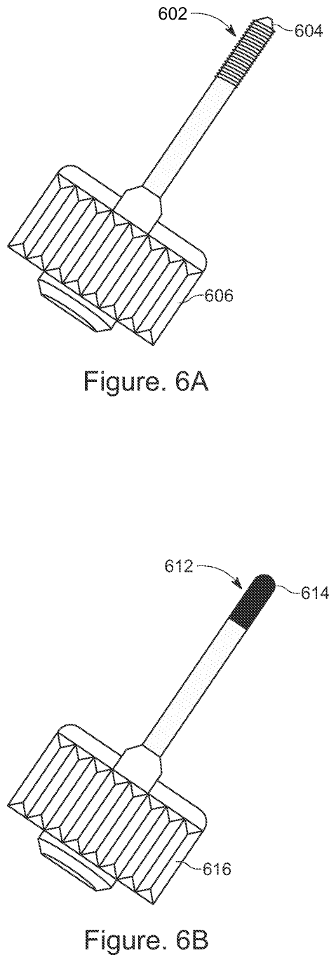

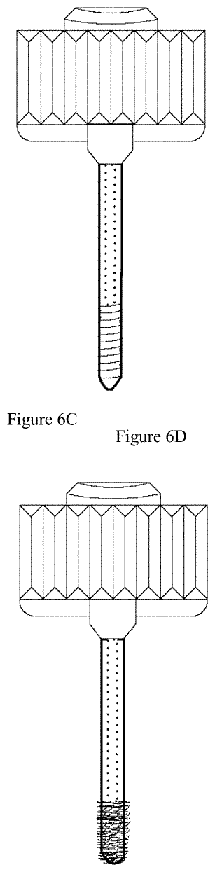

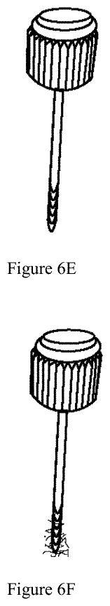

FIGS. 6A-6F illustrates a qube removal tool having sharp, jagged and/or barbed appendages for removing a qube from an oral surgical retraction site, or from a dental implant, or following use during oral surgery cushioning, bandaging, and/or protecting gum tissue at a bone graft site, a tooth extraction site, a dental implant site, or another oral surgical site in accordance with embodiments. An example qube removal tool may articulate such that the barbed ends of two appendages may be safely enclosed or sheathed or enfolded or interlocked in a “safety-on” position and may be actuated or articulated into a “safety-off” position such as to emerge to grab a qube for removal from a retraction site, or a space maintaining site or a tissue protection side or from a dental implant site or other oral surgical qube use site.

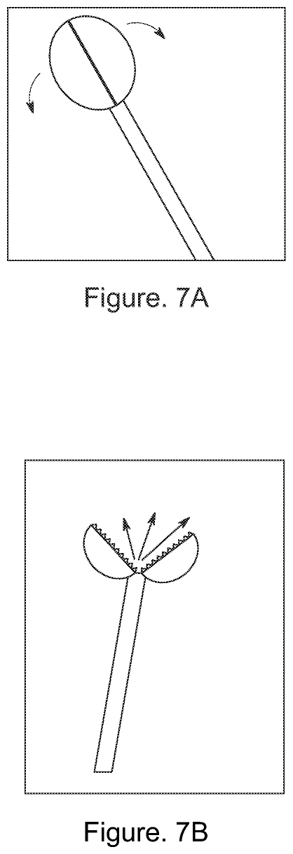

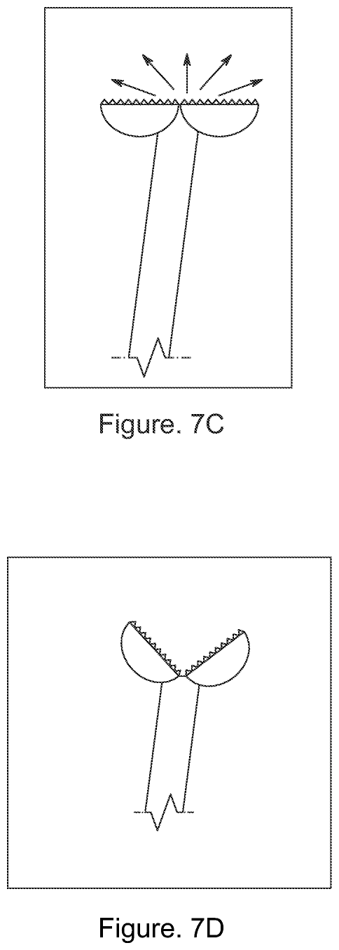

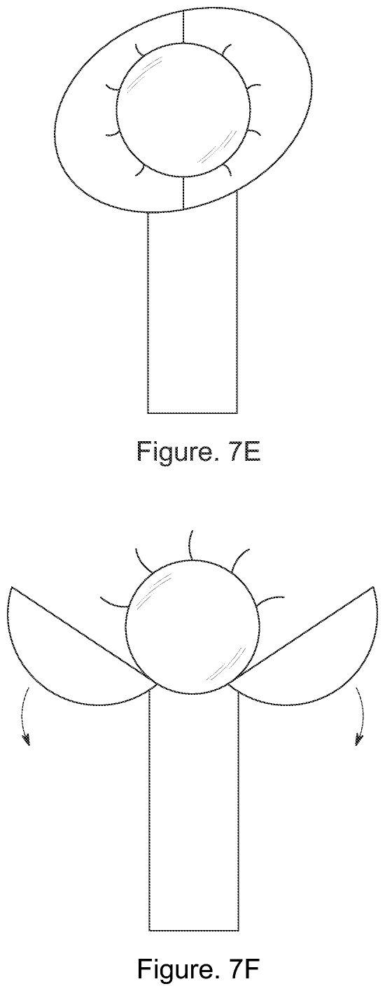

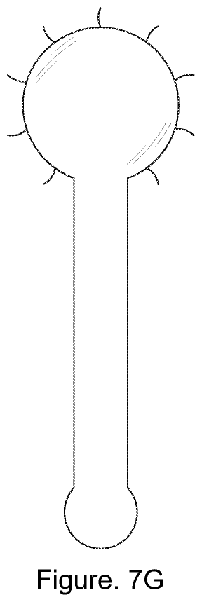

FIGS. 7A-7G illustrate a qube placement and/or removal tool that may have articulated arms or articulated ends or both for, respectively, pushing, maneuvering, reorienting or bluntly pinching or holding a qube for placement at an oral surgical site as a qube placement tool and/or for grabbing, entangling or adhering to a qube to remove it from an oral surgical site as a qube removal tool.

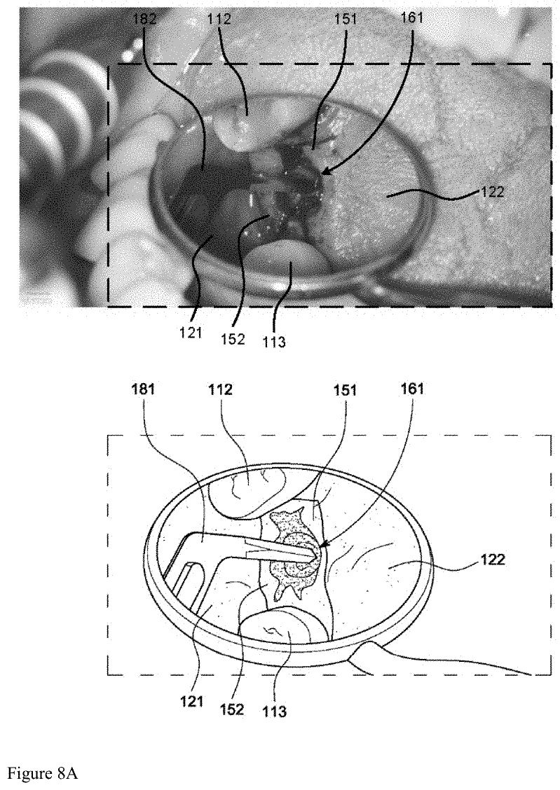

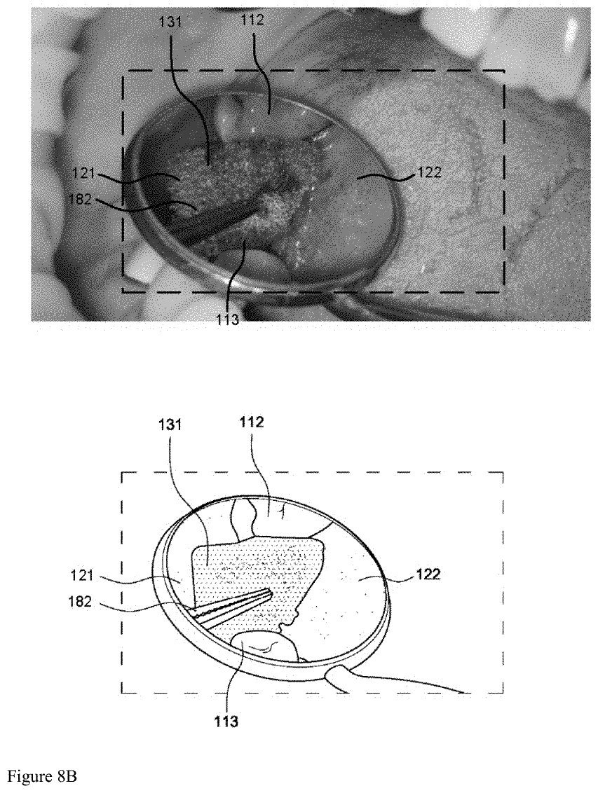

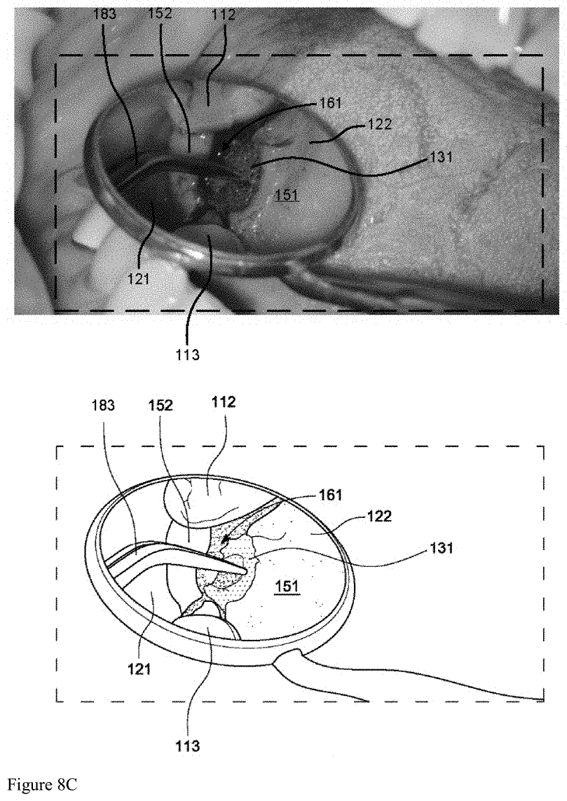

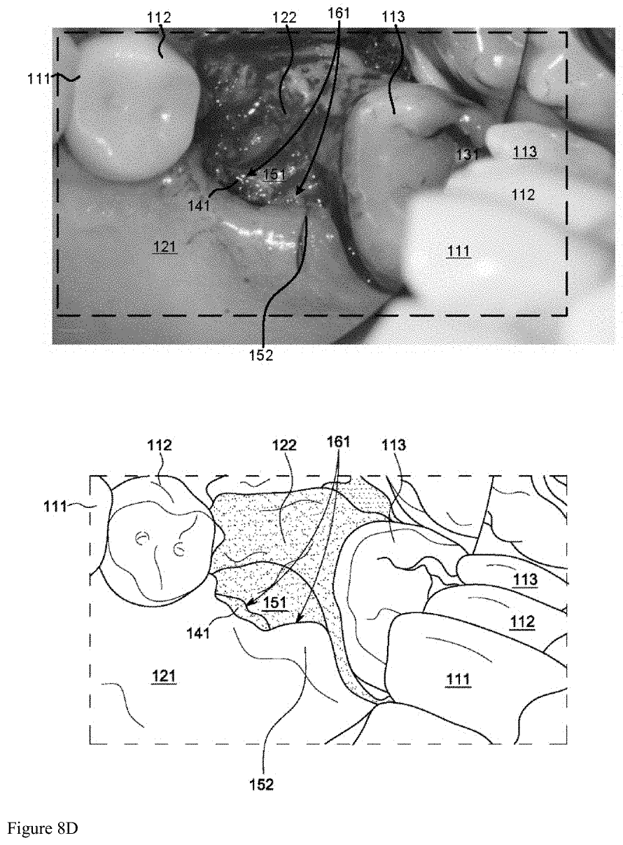

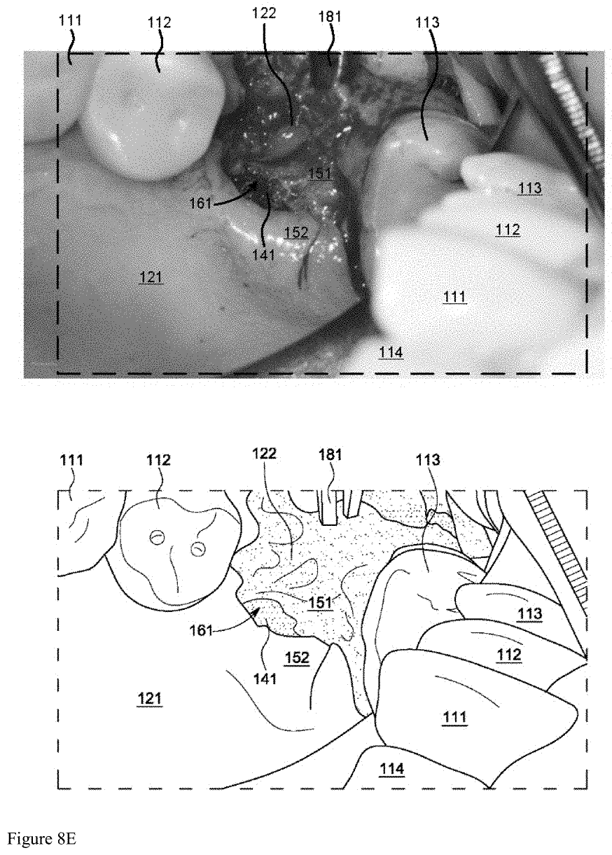

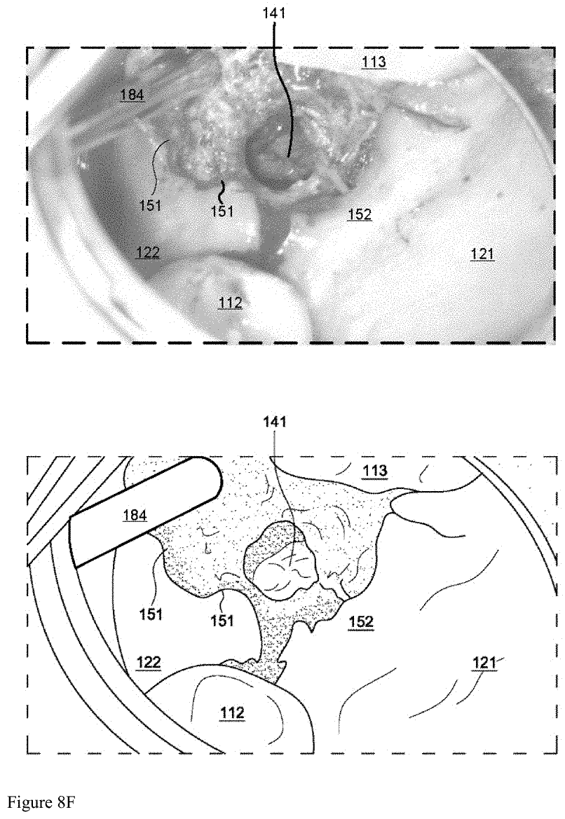

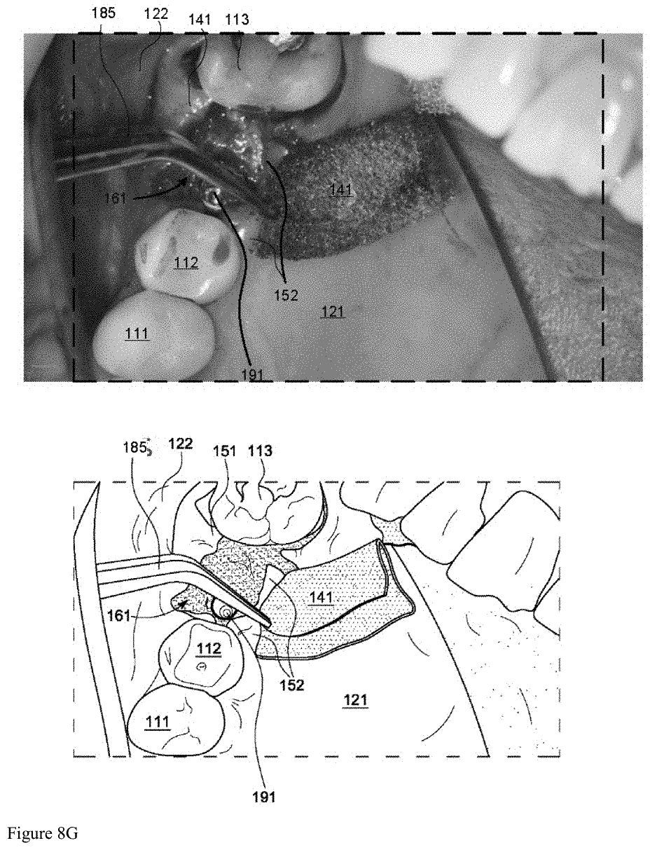

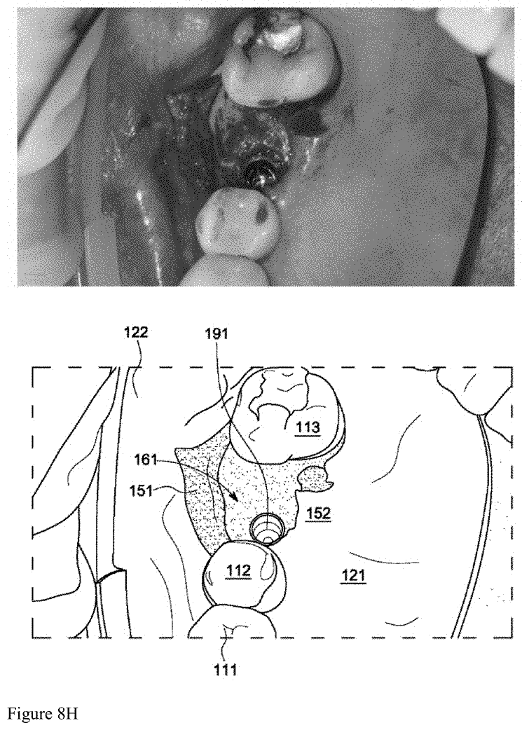

The photographs of FIGS. 8A-8I illustrate certain steps in a process leading incrementally to completion of the coupling of a dental implant at a site of tooth extraction, tooth absence, tooth loss or tooth decay. Advantageously, only minimal tissue trauma was caused by use of the qube.

Moreover, in certain example embodiments, use of a qube during an oral surgery or during a step or subset of steps of an oral surgery, e.g., a dental implant surgery, a tooth or jawbone grafting surgery, or another oral surgery involving one or more retraction uses of one or more cubes. In example embodiments, a dental impression may be made, formed, generated or located such as to make a dental impression for molding a synthetic tooth, a grown organic tooth or a tooth graft or set of teeth to replace a tooth or teeth that may have become decayed or that may be colliding with another tooth or gum, cheek, tongue or lip area causing pain or that may be rooted unevenly within an upper or lower jaw in the front or back of the mouth or may have fallen out such that a synthetic replacement tooth or a grown organic replacement dental implant or similar oral constituent may be desired to take its place.

Example embodiments are provided herein that may involve one or more oral surgical steps, sequences of two or more steps, subsets of multiple steps or several steps, or complete oral surgical processes that involve use of a qube for retraction, maintaining space above or within a dental implant, abutment or crown, or providing temporary structural integrity support for a tooth, gum, dentin, pulp, root, enamel, bone-cementum, crown or combinations or component parts thereof, or for catching, filtering, redirecting, accumulating, or stabilizing or controlling flow rate, area coverage or contained volume density of bodily fluids, saliva, blood, mucous, water, partly digested food or dislodged food fragments or combinations or evolving quantities or components thereof during an oral surgery.

Example embodiments may advantageously further involve reduced pain, reduced swelling, and reduced tearing, scratching, slicing, stabbing or poking by sharp edges or jagged components of dental instruments, and reduced time to heal and enhanced effectiveness by placement and use of one or more qubes for protecting, cushioning, deflecting, bandaging, or covering one or more exposed, wounded, inflamed or otherwise sensitive areas within a patient's mouth during an oral surgery.

Example embodiments of dental processes, both surgical and non-surgical, advantageously include sequences of steps involving use of one or more qubes for retraction, maintaining space, cushioning, absorbing, softening, providing flexibility, strength without rigidity, and cohesiveness. After any of a wide variety of oral surgical steps, and in various orders and sequences of oral surgical steps, use of qubes throughout the surgical processes characteristically maintains an availability of choices of next steps, when to stop, how to provide a first dental care process and transition to a different oral state prepared to provide a second dental care process, while continuously, discretely, periodically and/or increasingly having an ability to return, and/or returning, suturing or positioning or orienting tissue to an original position or orientation due to no distortion or damage being caused by this retraction method involving use of a qube rather than a conventional retraction cord or other conventional retraction device or component.

FIG. 8A includes a photograph that illustrates an incision site reflected in a mirror in accordance with an example embodiment.

FIG. 8B includes a photograph of a retraction qube inserted into an underside of a surgical flap for retraction in accordance with an example embodiment.

FIG. 8C includes a photograph of a qube further inserted under a flap in accordance with an example embodiment.

FIG. 8D includes a photograph including qubes placed on both sides of an incision to retract a surgical flap and expose underlying bone in accordance with an example embodiment.

FIG. 8E includes a photograph of a visible qube retracting a flap in accordance with an example embodiment.

FIG. 8F includes a photograph that illustrates retraction qube allowing access to osteotomy for implant site preparation and better visibility in accordance with an example embodiment. A floor of a maxillary sinus is visible in the photograph of FIG. 8F as the grey circular area in the image. Post-operatively, however, the patient had minimal pain, swelling and inflammation due to use of a qube retraction medium in an advantageous form of retraction during an oral surgery. There was also a strong unobstructed healing response due to lack of trauma during the surgery.

FIG. 8G includes a photograph wherein the retraction qube of FIG. 8F has been removed in accordance with an example embodiment.

FIG. 8H includes a photograph of a qube removed from an opposite side of an implant in place below a gingival surface in accordance with an example embodiment.

FIG. 8I includes a photograph of a surgical flap closed and sutured around a dental implant site in accordance with an example embodiment. In fact, a pair of surgical flaps are shown sutured in place on opposite sides of a dental implant site that includes an abutment component coupled to a dental implant that is secured to the jawbone of a dental customer or orthodontal patient. A crown may be next coupled to the abutment component of the example embodiment that is illustrated photographically at FIG. 8I.

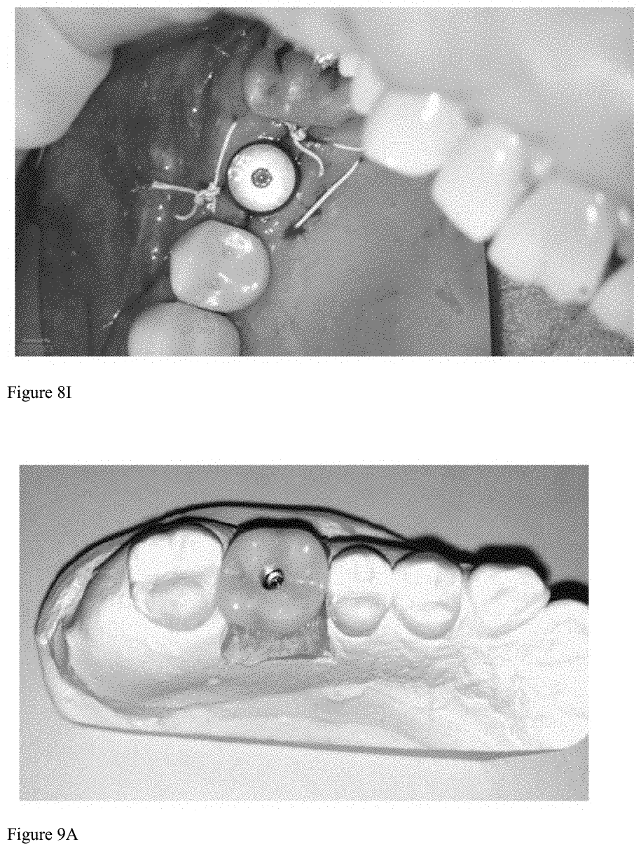

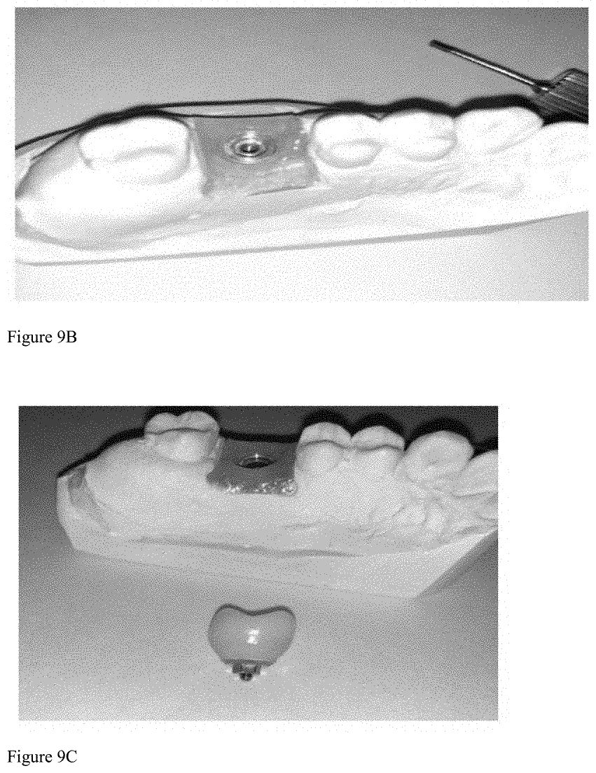

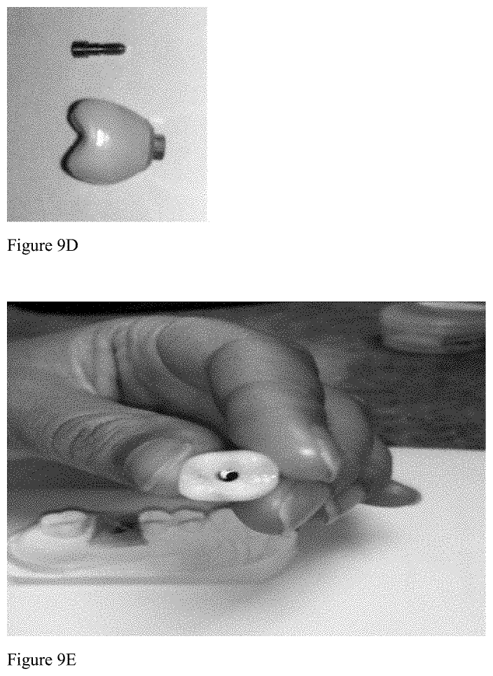

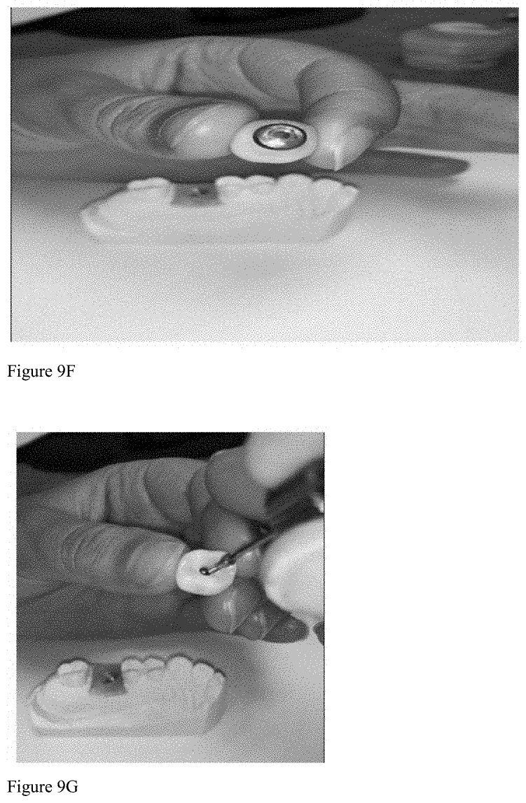

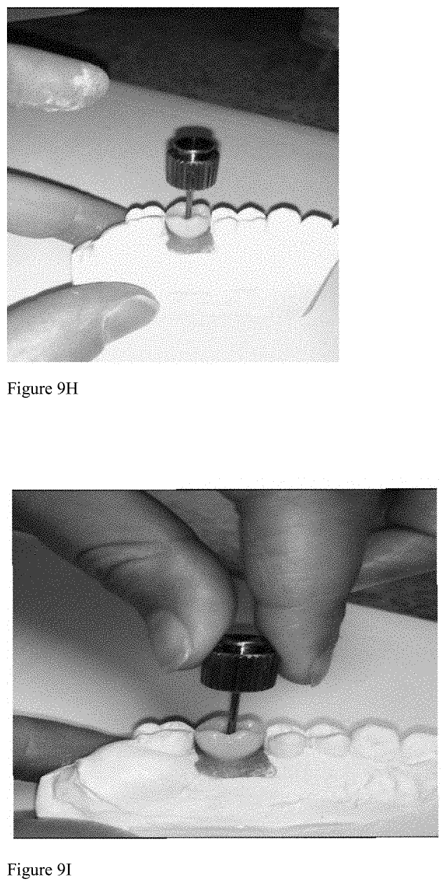

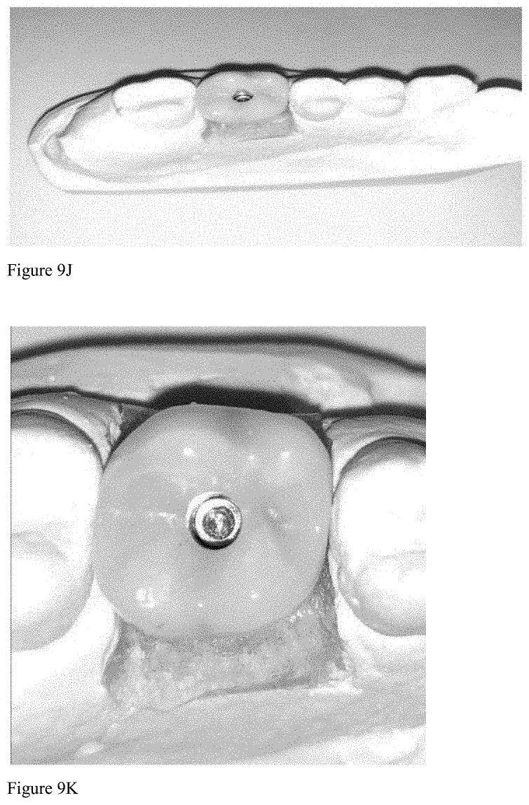

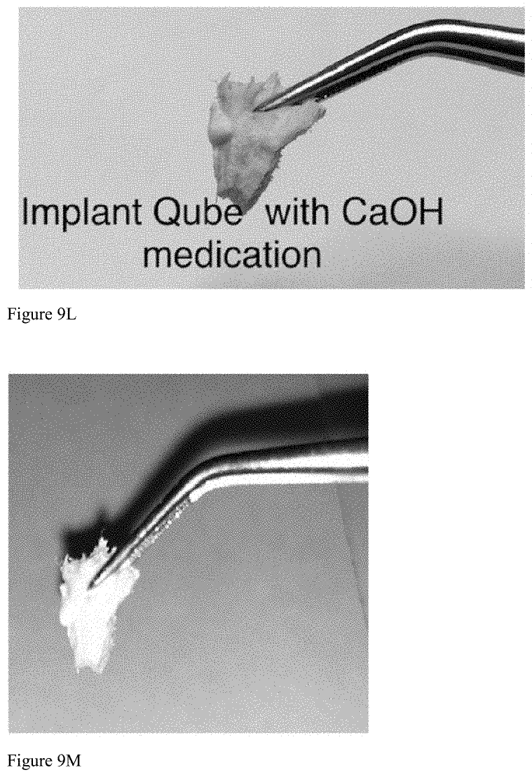

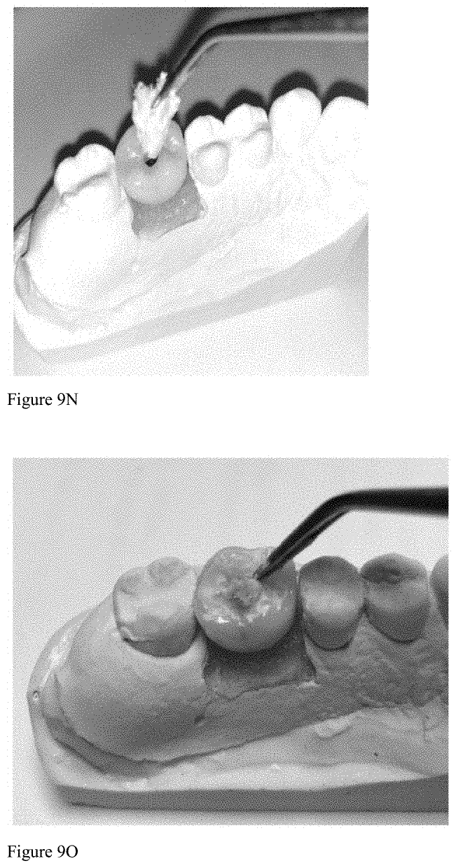

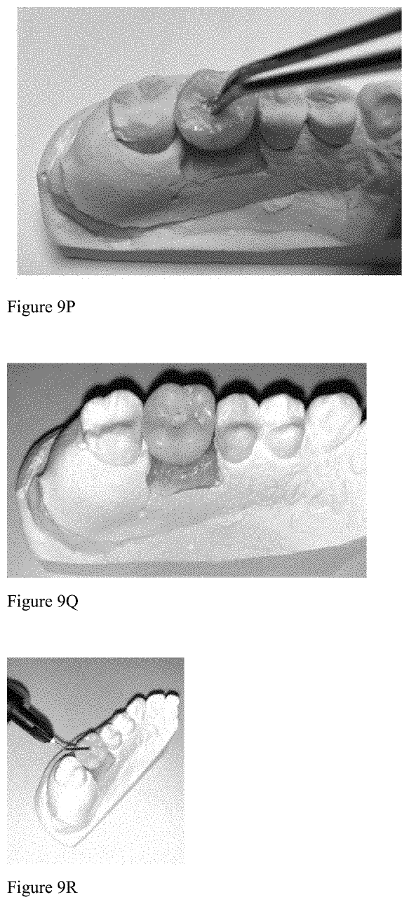

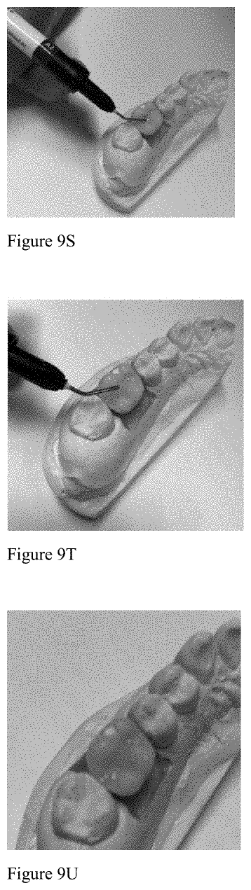

FIGS. 9A-9U illustrate photographically examples of endocubes and/or dental implant cubes in accordance with example embodiments.

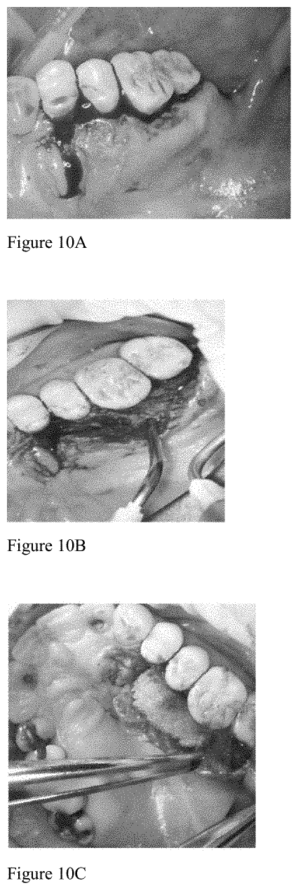

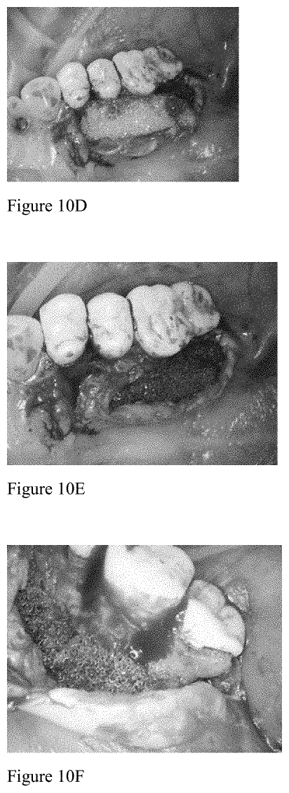

FIGS. 10A-10I include surgical photographs that include qubes in place and in use during performance of various oral surgical steps. In these example embodiments, the Qube material exhibits advantageous usefulness and functionality as a demonstrably suitable retraction medium.

FIGS. 10A-10I are photographs illustrating multiple uses of qubes of different sizes and shapes specifically configured for a planned use during one or more scheduled oral surgeries in accordance with example embodiments. FIGS. 10A-10H include multiple photographs that include one or more qubes each in place performing a retraction function. Other uses of qubes include performing a spacer or space-maintaining function during an oral surgical procedure that includes two or more subsets of an overall surgery or of a complete procedure, such as between coupling a dental implant at a grafted or ungrafted jawbone socket site which has become decoupled from a tooth suddenly or gradually over time, or a jawbone site that is at risk of becoming decayed unless a rotting tooth is extracted or repaired.

The two or more subsets of sequential oral surgical steps, processes, actions or modifications may, in one example embodiment, be spaced apart in time. In an example embodiment, a time delay advantageously allows for sufficient osseointegration of a bone graft within a jawbone socket, or socket graft, for example, prior to a dental implant procedure. Such a dental implant procedure may itself follow a sudden, unexpected tooth loss collision event or a long and steady incremental tooth decay process, or an ordinary tooth extraction, or a drawn-out, crumbling tooth disintegration lasting perhaps years or another tooth and/or jawbone volume reducing event.

The two or more surgical process subsets may, in another example embodiment, be spaced apart in time in order to allow sufficient osseointegration of a dental implant inserted within a jawbone socket at a depth below a gingival margin anywhere in a range between a shallow implant coupling location through an average implant depth location to a deep implant coupling location that may be significantly below a gingival margin. In this example embodiment, a second surgical process subset may involve coupling within a dental implant component for maintaining a space for attaching an abutment after sufficient osseointegration of the implant has occurred over the passage of time.

FIGS. 10A-10H include surgical photographs that include qubes in place and in use during performance of various oral surgical steps. In these example embodiments, the Qube material exhibits advantageous usefulness and functionality as a demonstrably suitable retraction medium.

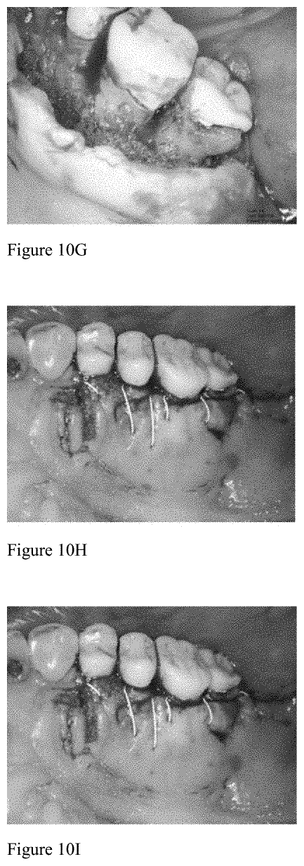

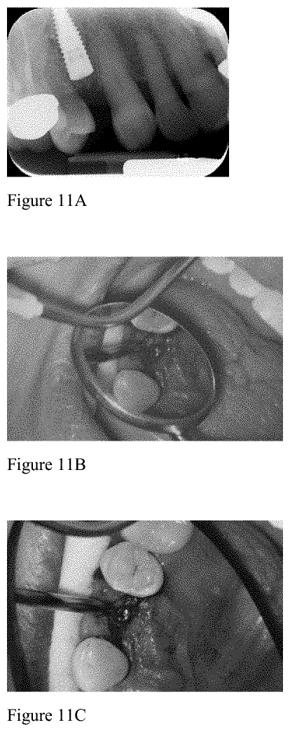

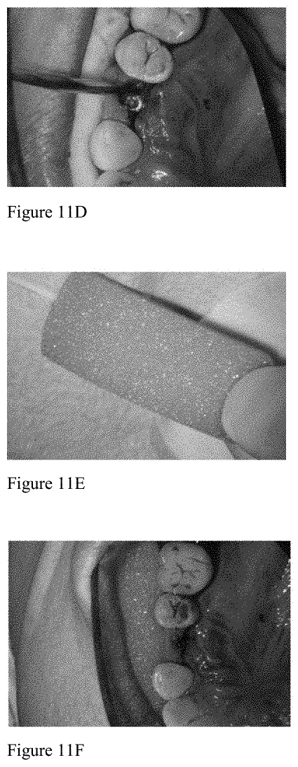

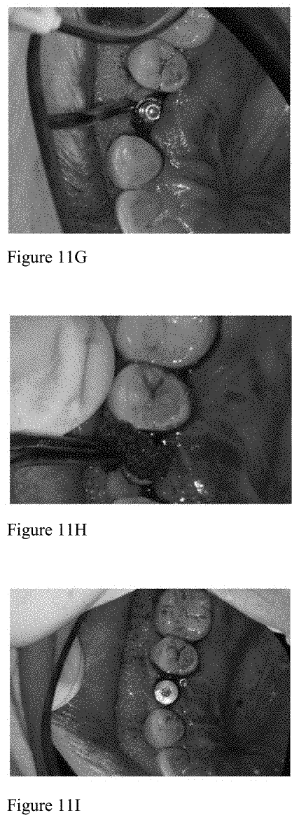

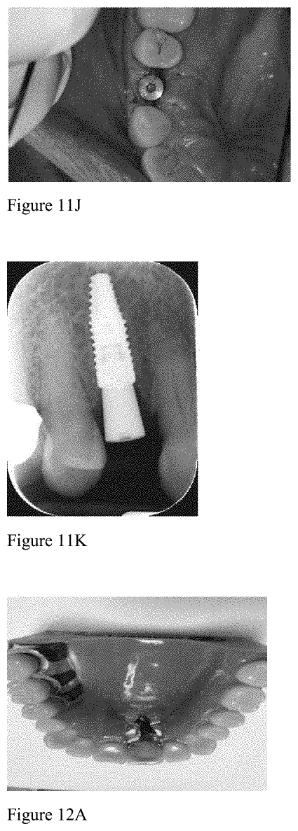

FIGS. 11A-11K illustrate example embodiments of example retraction steps and example retraction qubes useful in multiple roles during a dental implant procedure involving a screw-coupled dental implant in accordance with example embodiments.

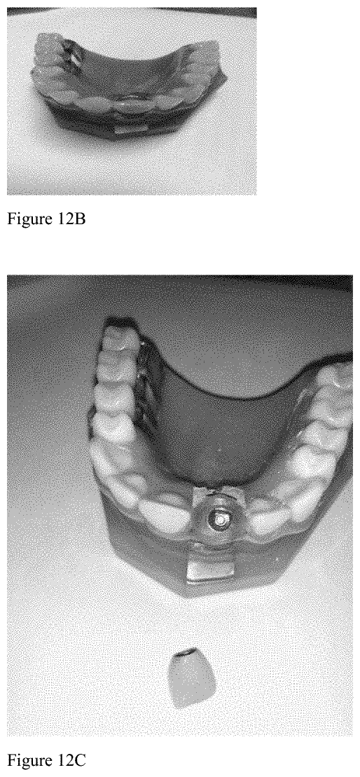

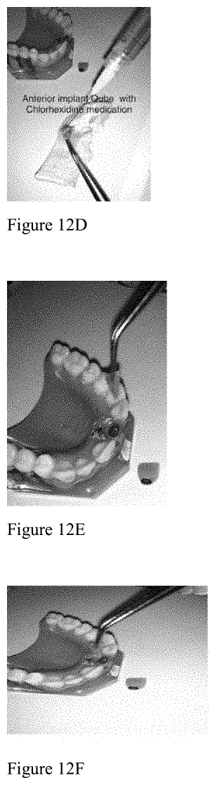

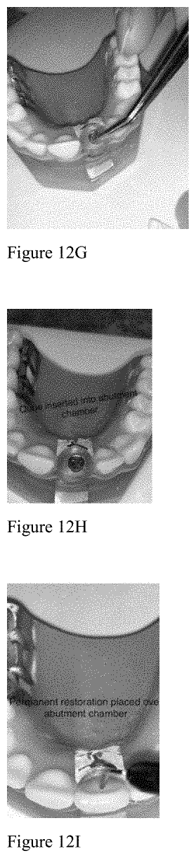

FIGS. 12A-12J illustrate example embodiments of example retraction steps and example retraction qubes useful in multiple roles during a dental implant procedure involving a cement-coupled dental implant in accordance with example embodiments.

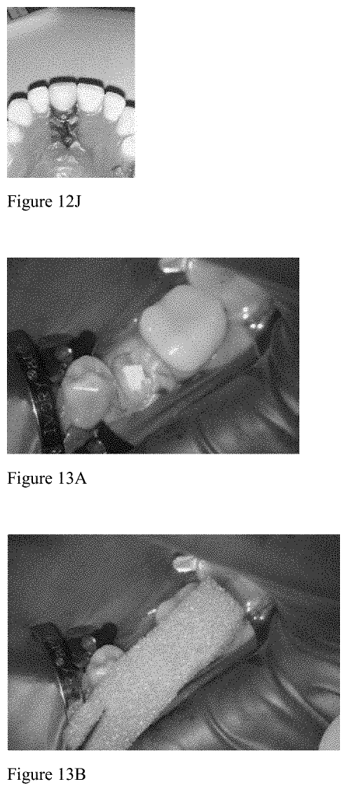

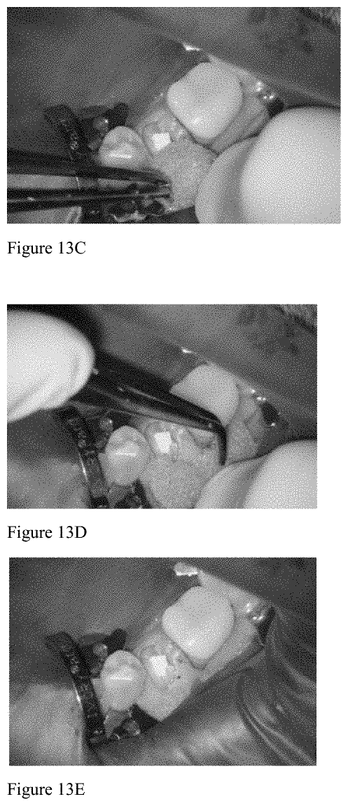

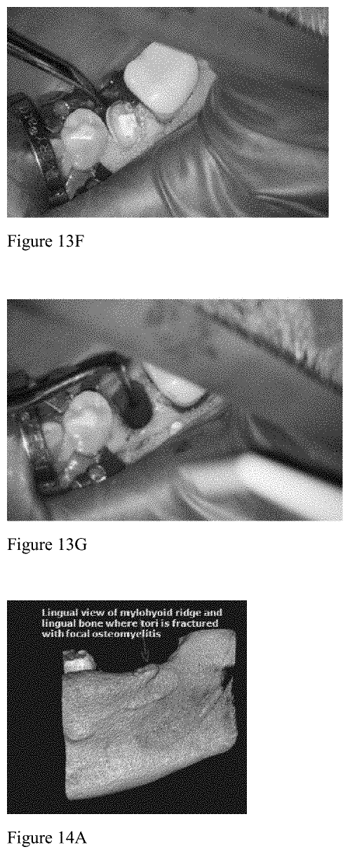

FIGS. 13A-13G photographically illustrate further example qube uses and applications and example qubes. One or more qubes may be used to retract a rubber dam. A qube may be used to retract soft tissue as well. A qube may be used to protect cheek tissue, tongue tissue, lip tissue, gum tissue, tonsil tissue, and/or tissue at the roof of the mouth or under the tongue from a surgical drill or other surgical instruments. A qube may protect tissues of the mouth from encountering tooth or implant fragments which may have sharp or jagged edges by adhering, blocking or deflecting such items.

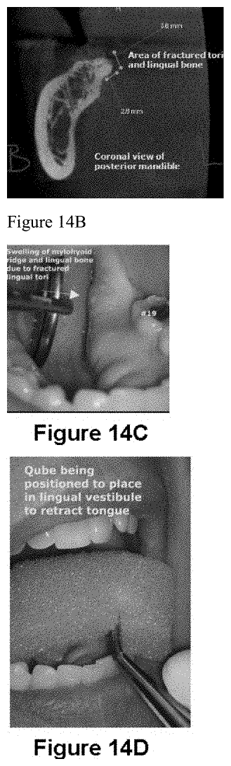

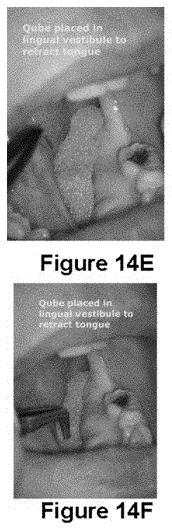

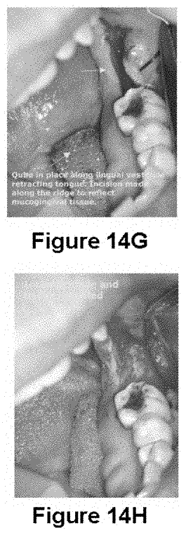

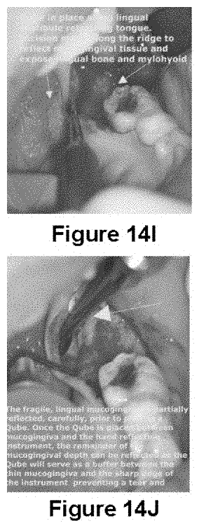

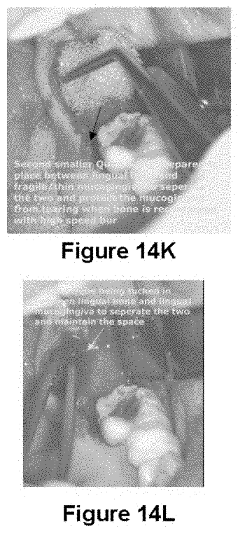

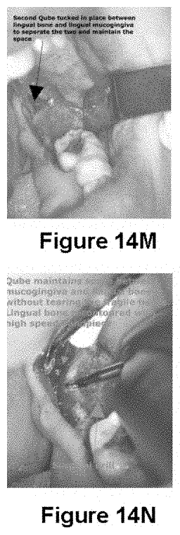

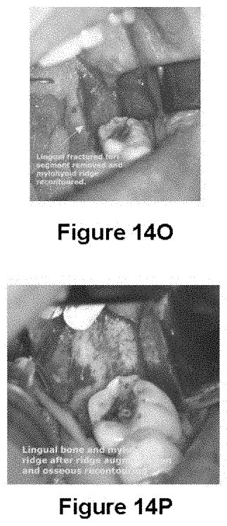

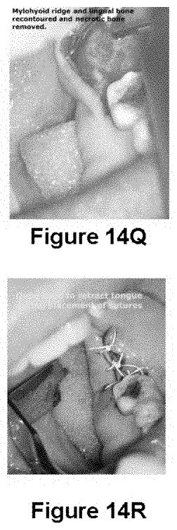

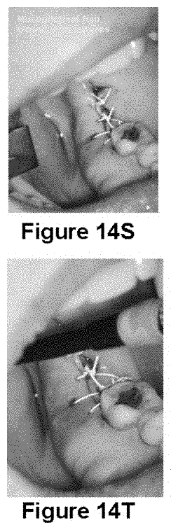

FIG. 14A-14T illustrate photographically an example procedure using retraction qubes in accordance with another embodiment.

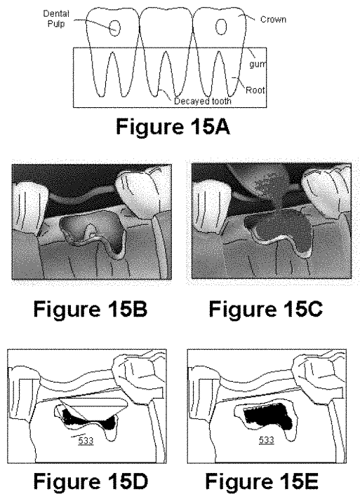

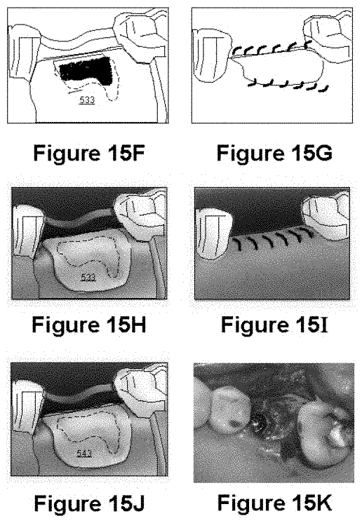

FIG. 15A schematically illustrates a decayed tooth 1512, which may also be a deformed tooth, a misplaced tooth, a misoriented tooth, a pain-producing tooth, an outsized molar or an otherwise unwanted tooth 1512, which is located between a pair of healthy teeth 1511, 1513, and which is prior to extraction of the decayed tooth 1512, or prior to a collisional tooth loss, a disintegrational or naturally decaying tooth loss, or another unintended tooth loss, in accordance with example embodiments.

FIG. 15B schematically illustrates the pair of healthy teeth of FIG. 15A following extraction or other loss of decayed tooth 1512 leaving a gap both between the healthy teeth 1511, 1513 above the gumline 1521 and extending beneath the gumline 1521 into a socket recess defined in a jawbone region from which a root region of the extracted decayed tooth 1512 of FIG. 15A has also been removed following incision and retraction of gingival flaps 1551, 1552 around the decayed tooth 1512 in accordance with an embodiment.

FIG. 15C schematically illustrates teeth with a gap above the gumline and a socket recess defined through the gumline and into the jawbone beneath after a decayed tooth extraction with the socket filled or partially filled with graft material in accordance with an embodiment.

FIG. 15D1 schematically illustrates teeth with a gap above the gumline and a socket recess defined as extending into the gum tissue and into bone tissue beneath with a qube 1533 draped over a graft-filled socket as in FIG. 15C to protect and promote osseointegration at the socket graft site and to cushion and bandage the gums around the socket for healing in accordance with an example embodiment.

FIG. 15D2 schematically illustrates teeth with a gap above the gumline and a socket recess defined as extending into the gum tissue and into the bone tissue beneath with a qube 1534 inserted or partially inserted into a partially graft-filled socket as in FIG. 15C to protect and promote osseointegration at the socket graft site and to cushion and bandage the gums around the socket for healing in accordance with an embodiment.

FIG. 15E schematically illustrates sutured gingival flaps 1571, 1572 following removal of retraction qubes 1531, 1532 to close a socket graft site draped with qube 1533 for osseointegration in accordance with an embodiment.

FIG. 15F schematically illustrates teeth with a gap following osseointegration, removal of sutures, incision and retraction again of gingival flaps 1551, 1552 at a socket graft site that is still protected by a blood-soaked qube 1543 in preparation for a dental implant procedure in accordance with an embodiment.

FIG. 15G schematically illustrates an osseointegrated socket graft site following removal of a blood-soaked qube 1541 in accordance with an embodiment just prior to coupling a dental implant into the socket graft site in accordance with an embodiment.

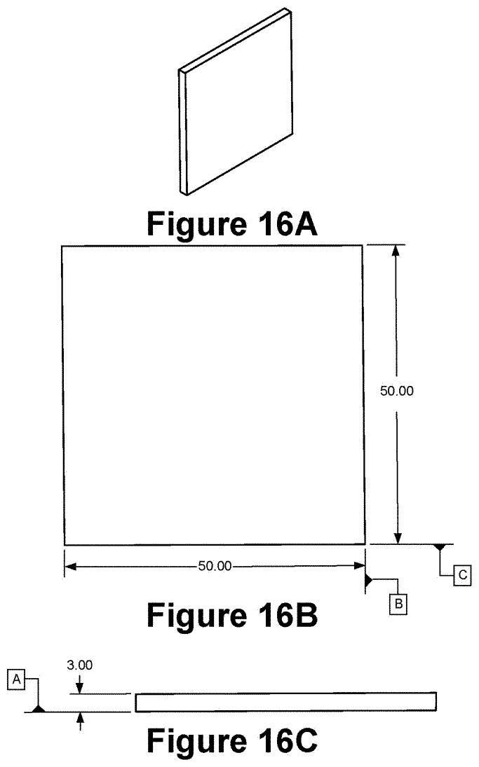

Example embodiments include the following five specific Qube embodiments that are illustrated schematically at FIGS. 16A-29C and described below with reference thereto. The dimensional ratios, overall sizes and volumes, surface areas, thicknesses and curvatures, and lengths of sides are illustrative, and additional embodiments may vary with regard to each of these and other parameters described herein.

A pore density of a qube may be selected in accordance with an application to which a qube is to be advantageously used. In example embodiments, a qube may also be selected that is advantageously either felted or unfelted, and in felted embodiments, the felting may be between two to one (2:1) and four or five to one (4:1 or 5:1), or between 2.5:1 and 3.5:1 or in a range around approximately 3:1. In a specific example embodiment, a 3:1 felted, 30-110 ppi, e.g., 60-90 ppi or 70-90 ppi, or unfelted 50 ppi-100 ppi, polyester polyurethane, polyether polyurethane and/or polytetrafluoroethylene (PTFE) expanded foam qube may be used as a gauze replacement in various applications to which cotton gauze is conventionally used.

In different applications, pore densities of example embodiments of qubes may include between not less than 45-50 pores per inch (ppi) and perhaps not greater than 100-105 ppi. Beneath 45 ppi, e.g., around 40 ppi, structural integrity may become an issue, while above 105 ppi, e.g., around 110 ppi, absorption and fluid transmission using suction become too low to be useful for this application and certain other applications. For example, when serving as a suction filter to draw excess blood and saliva through as part of a dental procedure, qubes between not less than 45 ppi and 75 ppi or perhaps more if desired for other reasons, or between 45 ppi and 70 ppi, or between 50 ppi and 65 ppi or between 55 ppi and 60 ppi may be selected. A cylindrical 3D mushroom shaped qube or a 2D mushroom shaped qube may be used for compacting graft material at a tooth extraction site and/or for suctioning blood and/or saliva therethrough without disturbing the grafting material and in certain embodiments serving to compress the grafting material by contact and application of pressure. Multiple applications, e.g., two or three or four or five, of supplying loose grafting material at a tooth extraction site may be performed sequentially each involving compacting the grafting material by contacting and applying pressure to the graft material with a mushroom shaped qube.

A U-shaped qube may be installed at a tooth extraction site for allowing a patient to bite down comfortably to stop the bleeding and/or to compress loose grafting material without the U-shaped qube adhering to the grafting material and pulling it out when the U-shaped qube is removed. In each case, excess blood and/or saliva may be drawn or siphoned or suctioned through a qube which may contact graft material without pulling the graft material out of the socket nor otherwise loosening a compressed grafting pile when the qube is withdrawn or removed. Examples of U shaped qube embodiments may include felted polyester polyurethane or polyether polyurethane that are between 2:1 and 4:1 or 5:1 felted, e.g., approximately 3:1 felted, and between not less than 45-50 ppi and not more than 100-105 ppi, or between 60 ppi and 90 ppi, or between 70 ppi and 80 ppi, or between 70 ppi and 75 ppi, or approximately 72 ppi.

Another example embodiment may include a polyether polyurethane foam qube between 65 ppi and 90 ppi that may be selected for grafting applications when enhanced durability is desired.

In another example embodiment, a polyester polyurethane qube may have a pore density in a range between not less than 45-50 ppi and not more than 70-75 ppi or between 55 ppi and 65 ppi or between 57.5 ppi and 62.5 ppi or between 59 ppi and 61 ppi or within another range around approximately 60 ppi.

In the context of use of qubes to apply compression at a wound site, a felted qube including between 70 ppi and 105 ppi or between 80 ppi and 100 ppi or between 85 ppi and 95 ppi or another range around 90 ppi may be selected. In the context of use of qubes as wound disinfection tools, e.g., at a tooth extraction site, example embodiments may include felted qubes that include pore densities between not less than 45-50 ppi and 70-75 ppi.

As a retraction instrument, an example embodiment may include a rectangular qube with a pore density between 55 ppi and 85 ppi, or between 60 ppi and 80 ppi, or between 65 ppi and 75 ppi or another range around approximately 70 ppi. In other example embodiments, retraction qubes having lower pore densities between 40 ppi and 70 ppi, felted or unfelted, may be selected.

SG Series Qube

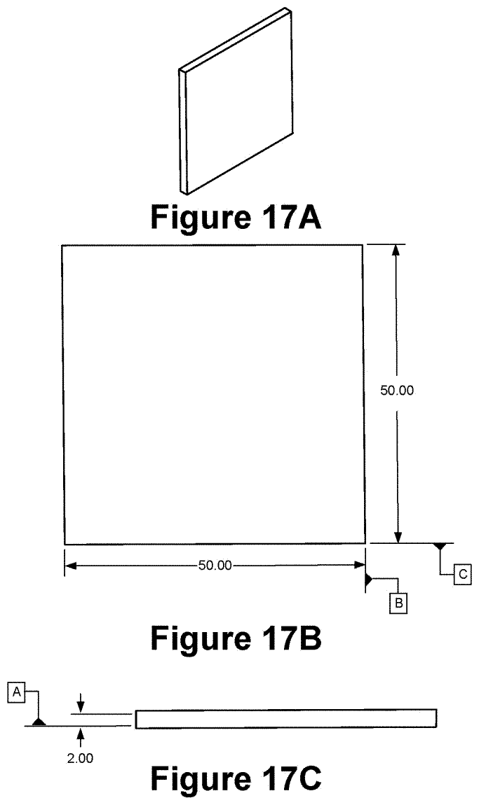

The “Super Gauze” Qube or SG series qube may be used for applications in place of traditional cotton gauze. Super Gauze may be square shaped, e.g., 50 mm×50 mm sheets that may be 1-10 mm thick, including 2 mm or 3 mm in thickness as in the examples of FIGS. 16A-16C or 17A-17C, respectively. Other examples include 25 mm×25 mm or smaller, 75 mm×75 mm, and 100 mm×100 mm or larger. A qube can be formed as most any shape, diameter or thickness, depending on the application.

An SG series qube may be advantageously used for applications where traditional 2″×2″, 3″×3″ or 4″×4″, for example, cotton gauze pads are conventionally used such as for absorbing blood and saliva from wound sites in the oral cavity or externally applied to skin wounds. Lots of teeth are extracted and the SG product can easily be substituted for cotton gauze and applied to extraction sockets to initially compress the surface of the wound for initial clotting. Doctors typically fold the flat cotton gauze to their desired shape and size and then the patient bites down on the wad of gauze. This example is for typically a short time usage during a dental procedure. The Super Gauze product does not adhere to the surface of wound sites like cotton gauze often tends to do, especially after compressing the wound for initial hemostasis. So when a qube is removed from a wound site, unlike cotton gauze, a qube does not tend to pull away the initial fibrin clot that forms. In addition, qubes tend to absorb fluids including blood and saliva and can be easily wrung out to reuse immediately. The pore size and reticulated nature of the foam material of an example qube allows for material to be suctioned during a dental procedure to draw out blood or saliva from an oral surgical site, while the super gauze qube may be left in place to protect the wound. The material of an example qube can be rolled, folded or used in multiple layers depending on the procedure at hand.

C Series Qube

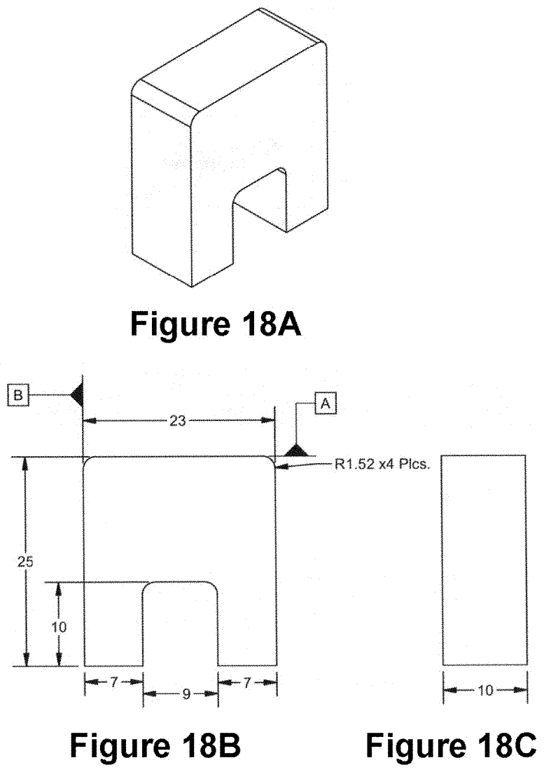

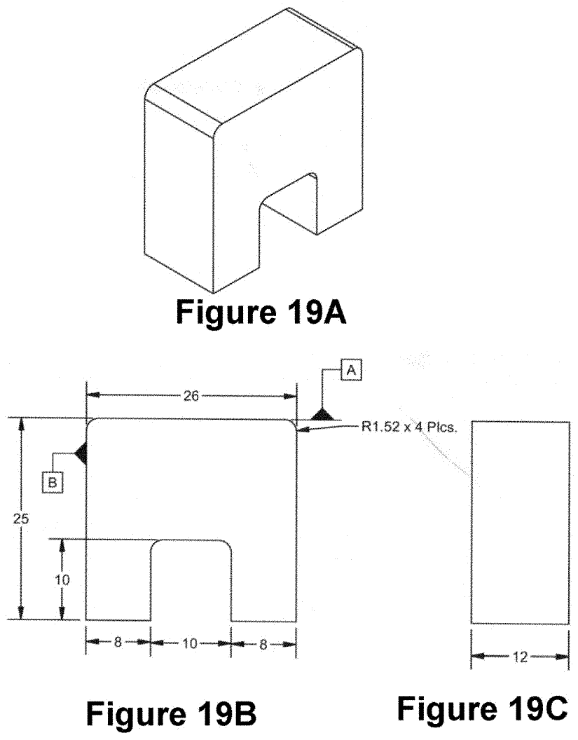

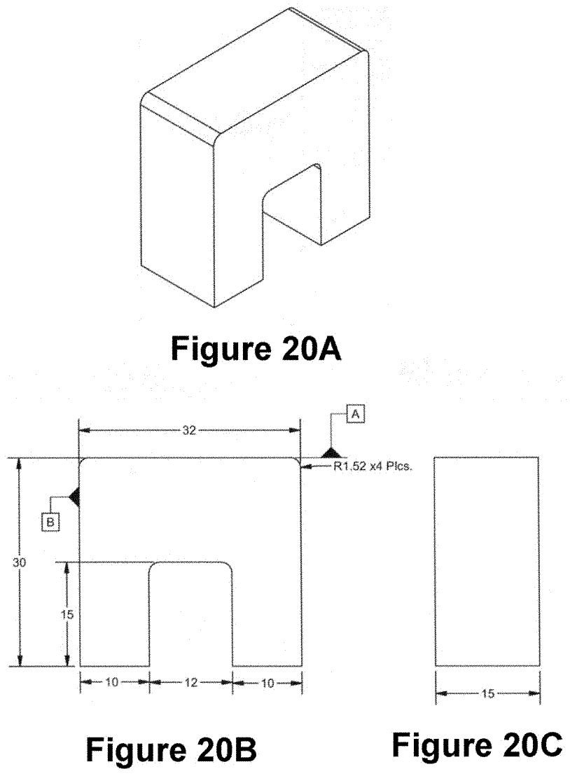

The “Compression” Qube or C series qube may be used advantageously as a cotton gauze replacement and can be used to decompress a wound site and stop bleeding after a tooth extraction or other surgery. This is a very common use of cotton gauze now. The horseshoe or U-shaped Qube product illustrated schematically in FIGS. 18A-18C, 19A-19C and 20A-20C, respectively, may be provided individually and/or in kits that include two or more sizes, such as three sizes: Small as in FIGS. 18A-18C (for the anterior teeth region), Medium as in FIGS. 19A-19C (for the premolar teeth region) and Large as in FIGS. 20A-20C (for the molar teeth region).

The unique shape allows for precise placement and firm retention over the extraction socket. The Compression Qube shape allows it to hug the topography of the alveolar ridge of both the maxilla and mandible when it is placed. The pore size and nature of the foam material of an example C series qube also allows for the material to further compress and pressurize over the tooth extraction site when the patient is advised to close/bite down for an initial 20-25 minutes. The additional compression action reapproximates the soft tissue and bone of the tooth socket (which have been distended following tooth extraction) back to or close to their original positions which reduces post-operative pain and ultimately expedites healing. The foam material of an example C series qube does not adhere to the surface of wound sites like cotton gauze tends to do, especially after compressing the wound for initial hemostasis. So when a qube is removed, unlike cotton gauze, the qube does not tend to pull away the initial fibrin clot that forms. This reduces prolonged bleeding. This also helps form and protect a very stable fibrin clot which serves as the foundation for primary wound healing, e.g., of a tooth extraction socket. In addition, an example C series qube may absorb fluids including blood and saliva and can be easily suctioned out while in an oral cavity.

G Series Qube

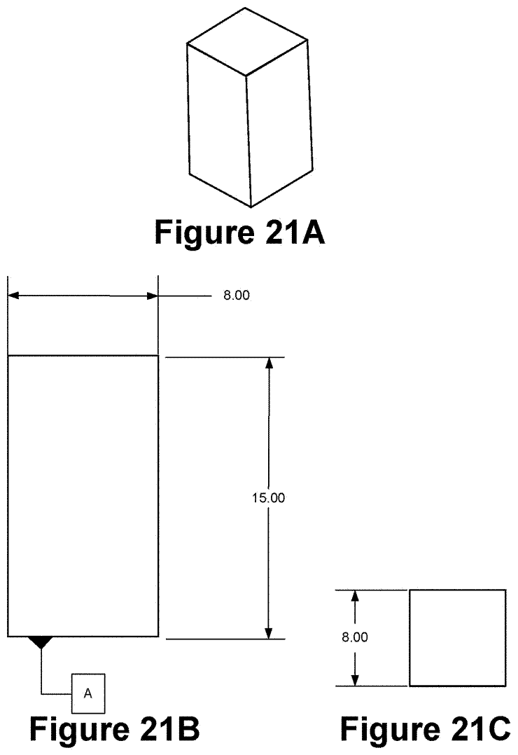

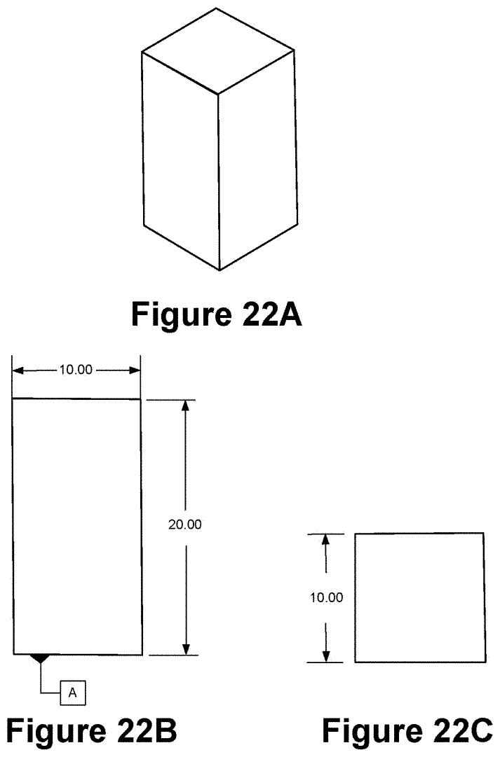

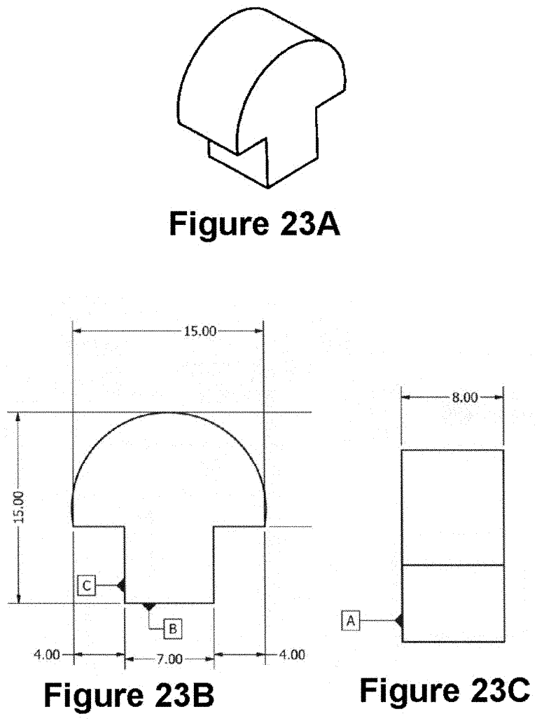

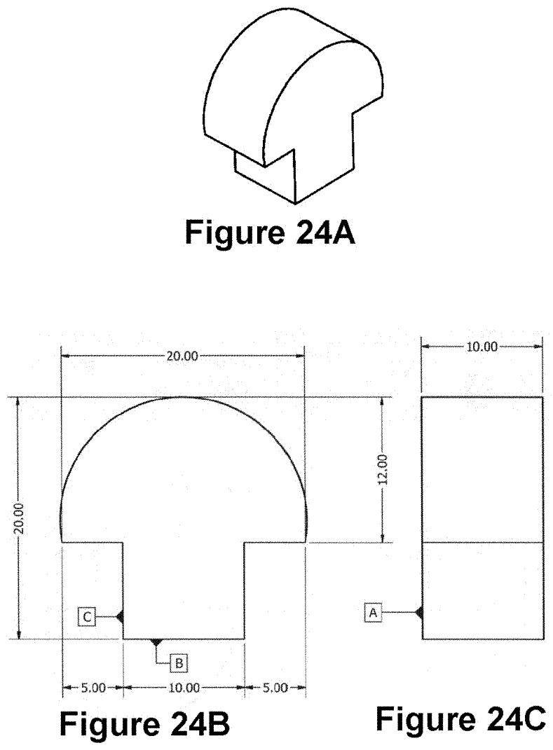

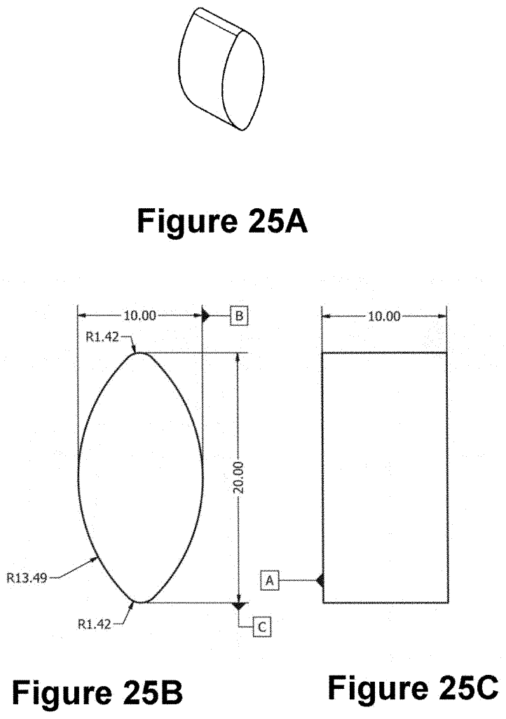

A Grafting Qube or G series qube may be used as an aid for the placement of bone graft material into a socket on an oral cavity where a tooth has been extracted and where new bone is desired to be set in the oral cavity in order to do a future tooth replacement. Currently, cotton gauze is what is mainly used as an aid in this regard. Different shapes and sizes may be used. FIGS. 21A-21C and 22A-22C schematically illustrate examples of G series foam rectangular qubes of relatively small and large sizes, respectively. FIGS. 23A-23C and 24A-24C schematically illustrate examples of G series 2D foam mushroom qubes of relatively small and large sizes, respectively. FIGS. 25A-25C schematically illustrate an example of a G series 2D foam football qube. Other shapes and sizes may be used in further example embodiments.

Placement of bone graft material (e.g., human allograft, xenograft, autograft or alloplast synthetic graft material) can be completed immediately following tooth extraction (socket preservation) or grafting procedures that are completed at a later time such as ridge augmentation, lateral and vertical sinus lifts, and block grafting to name a few. Conventional graft materials have a consistency of ground up corral particles or large particles of sand, which is often mixed with a liquid to serve as a carrier such as sterile saline or blood particulate from the patient, PRP mixtures, PRF mixtures, Emdogain or other growth factors. During graft placement, the graft site may also be bleeding as this is actually desired for successful outcome as the bleeding graft site may be relied upon to provide a majority of growth factors and cellular components for proper wound healing and regeneration of new bone formation. There can also be background saliva in the field attempting to enter the site as well which is essentially a contaminant. Once the graft site receives a bolus of bone graft material that is delivered with a surgical spoon or other tool, the Qube can be advantageously used to help hold the powdered bone material in place, rather than quickly washing out of a tooth socket from blood and saliva. The doctor may be trying to create new bone in an area where a tooth was pulled and will eventually be replaced by an implant screw and crown that is screwed into the jaw.

The shape, size and proportions of a Grafting Qube or G series qube may be advantageously selected for its use as a tool and ergonomic handling ability.

MS Series Qube

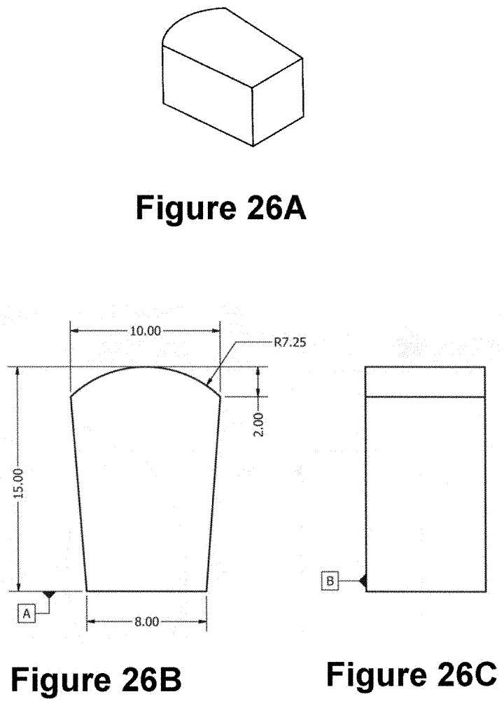

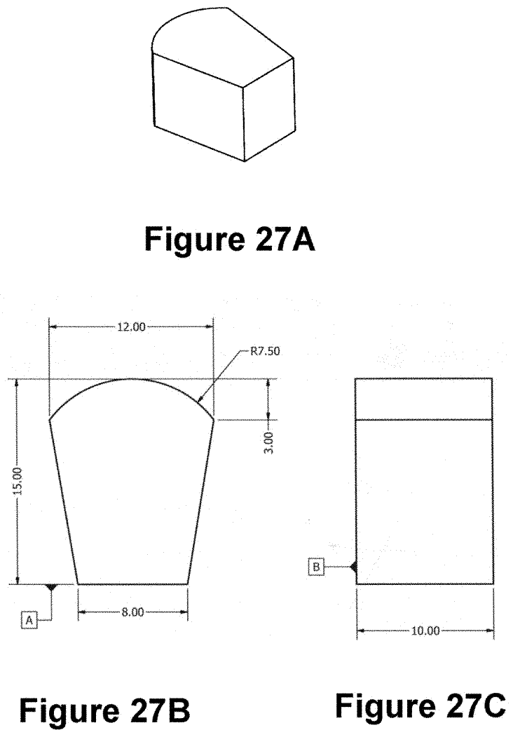

An MS series qube may have a 2D thimble shape and may serve advantageously as a cotton gauze replacement providing a convenient shape for surgical or non-surgical medical use when cleaning a skin area or other bodily area or applying a sterile or sterilizing solution, a medicament or absorbing or moving excess fluids. This 2D thimble shape may be an option for use in a grafting procedure, as a G series qube. The thimble shape may be 2D or 3D and may be made available in a variety of sizes, such as the relatively smaller and larger sized examples of FIGS. 26A-26C and 27A-27C, respectively. The radii of curvature of the top surfaces in the examples of FIGS. 26A-26C and 27A-27C are 7.25 mm and 7.5 mm, respectively, although these radii may be more or less than in these examples, such as 4 mm, 5 mm, 6 mm, 7 mm, 7.75 mm, 8 mm, 9 mm or 10 mm or more. The smaller radii of curvature would accompany smaller widths and the larger radii would accompany larger widths, at least at the top, whereas more or less tapering top to bottom than 20% or 33%, as in the illustrated examples, may be provided in further example embodiments. The base of an MS series qube may be square as in the example embodiment of FIGS. 26A-26C or rectangular as in the example embodiment of FIGS. 27A-27C. In other example embodiments, the base may be trapezoidal, circular, elliptical, triangular, pentagonal, hexagonal or another polygonal shape and/or a shape having one or more sides that are partially-curved or wholly-curved, convex or concave, or other regular or irregular shapes.

R Series Qube

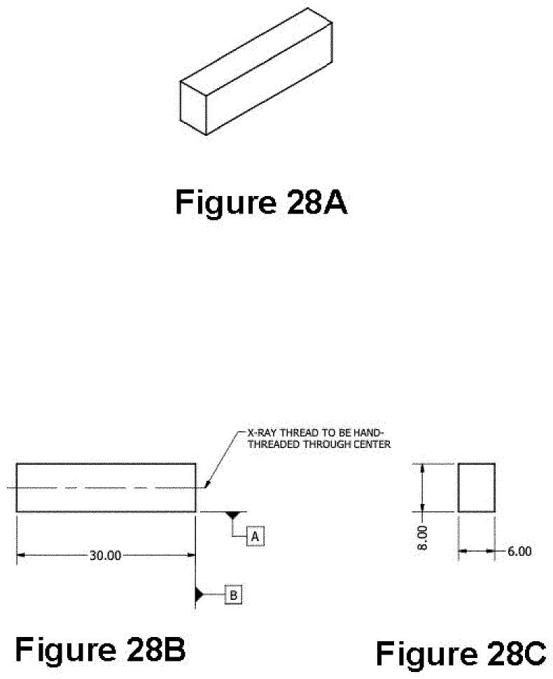

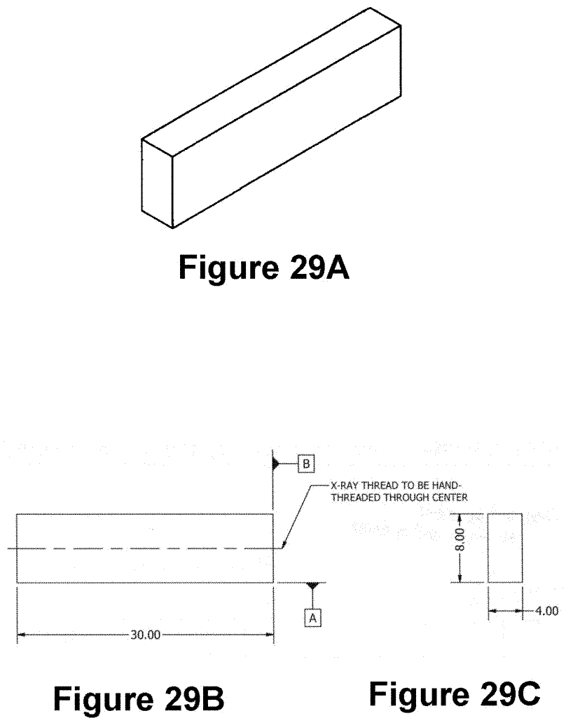

Example embodiments of relatively larger and smaller sized cotton gauze replacement qubes are, respectively, illustrated schematically at FIGS. 28A-28C and 29A-29C. The cotton gauze replacement qubes can be used instead of mechanical means or the insertion of cotton gauze on a temporary basis, e.g., to help keep a jaw bone and a patient's gum material separated during a surgical procedure. In an example embodiment, a thin rectangle shaped R series qube may be unfelted, although in further example embodiments, thin rectangle or rounded elongated qubes may be felted or unfelted.

These thin rectangle or otherwise elongated R series shapes may be configured in a variety of shapes, sizes and proportions. For example, example embodiments may have symmetric cross sections such as squares, circles or squares with rounded corners, or asymmetric cross sections such as ellipses, rectangles, rectangles with rounded corners, other polygons, or shapes including one or more convex or concave sides with or without rounded corners. In further example embodiments, the length may be selected to match a depth of a patient's jaw bone which may be more or less than the 30 mm shown in the examples of FIGS. 28A-28C and 29A-29C. Thicknesses smaller than 4 mm, such as 1 mm, 1.5 mm, 2 mm, 2.5 mm, 3 mm, or 3.5 mm may be provided in example embodiments, and thicknesses larger than 6 mm may be provided, such as 7 mm, 8 mm, 9 mm or 10 mm or more, and thicknesses between 4 mm and 6 mm may be provided such as 4.5 mm, 5 mm or 5.5 mm. The height may be more or less than the 8 mm shown in FIGS. 28A-28C and 29A-29C, in further example embodiments, as well, such as between 3-4 mm and 15 mm or more. The R series foam rectangular qube examples illustrated schematically in FIGS. 28A-28C and 29A-29C may include an x-ray thread that may be hand-threaded, or alternatively machine threaded, through the long dimension of the qube or such x-ray thread may be adhered to a surface of the qube or such x-ray thread may be formed through the qube during a curing process or poured and cured or a cavity formed through an R series qube may be provided to fill after curing with an x-ray thread or other x-ray process-specific material.

Each of the SG, C, G, MS and R series qubes may be available in any or all of broad ranges of sizes, shapes and/or proportions similar to the examples described with reference to the R series qube or otherwise.

While the invention has been described in terms of several example embodiments, those skilled in the art will recognize that the invention can be practiced with modification and alteration. The description is thus to be regarded as illustrative.

For example, further oral surgical or non-surgical dental applications of qubes may include root canal surgeries, teeth straightening, and teeth whitening. Example embodiments of qubes may be formed with PTFE, polyester polyurethane or polyether polyurethane in this and other contexts or with different materials such as those identified above herein or others with one or more similar or equivalent properties or characteristics.

There are also many practical applications in addition to dentistry wherein conventional use of cotton gauze may be advantageously replaced with the use of qubes as set forth herein or as may be modified to suit those particular uses. Such applications may include sutures, sponges, retractors and other items used in certain medical bodily surgeries other than oral surgeries, and first aid items such as bandages and wraps, and swabs, medicinal or cosmetic applicators, sponges and bodily fluid filters. The material forming a qube in alternative embodiments may include polyurethane additives such as hydroperoxide, bronze powder, isothiazolinone, zinc pyrithinone, thiabendazole, silver, quaternary ammonium, 10,10′oxybisphenox-arsine (OBPA), silicon oil, silicone surfactant, polyether, polyester, polyethyl, polyvinyl alcohol, or polydibutyltitanate. Hydroxyl values may be varied, e.g., between 27-58 mg/g. Acid values may be varied, e.g., between 0.05-0.08 mg/g. Water values may be varied, e.g., between 0.01-0.10%. Viscosity values may be varied, e.g., between 400-1225. Qube shapes can be modified in example embodiments by rounding corners that form right angles or otherwise smoothing sharp edges and/or corners.

A kit may include one or more of multiple types of qubes such as two of more of SG, C, G, MS and R series qubes may be included in the kit, and all five series types may includes one or two or three or several qubes. The qubes of each series type may be provided in different colors to easily distinguish them and/or may be provided in drawers or containment sections of the kit that are separated and labelled by series type and/or by specific applications or uses.

In addition, in methods that may be performed according to embodiments described herein and that may have been described above, the operations have been described in selected typographical sequences. However, the sequences have been selected and so ordered for typographical convenience and are not intended to imply any particular order for performing the operations, except for those where a particular order may be expressly set forth or where those of ordinary skill in the art may deem a particular order to be necessary.

A group of items linked with the conjunction “and” in the above specification should not be read as requiring that each and every one of those items be present in the grouping in accordance with all embodiments of that grouping, as various embodiments will have one or more of those elements replaced with one or more others. Furthermore, although items, elements or components of the invention may be described or claimed in the singular, the plural is contemplated to be within the scope thereof unless limitation to the singular is explicitly stated or clearly understood as necessary by those of ordinary skill in the art.

The presence of broadening words and phrases such as “one or more,” “at least,” “but not limited to” or other such phrases in some instances shall not be read to mean that the narrower case is intended or required in instances where such broadening phrases may be absent.

1. An oral surgical retraction article, comprising a polyester or polyether polyurethane foam sponge, or a combination thereof, that is autoclavable at 250° F. and has a porosity between not less than 45 pores per inch (ppi) and not more than 105 ppi and configured in size and shape for retracting a gingival flap during an oral surgery.

2. The dental surgical retraction article of claim 1, wherein said polyester or polyether polyurethane foam sponge includes an elongated shape.

3. The dental surgical retraction article of claim 2, wherein said polyester or polyether polyurethane foam sponge includes a cylindrical, ellipsoidal, tubular, wedge, prism, ovoid, triovoid, egg or pear shape, or combinations thereof.

4. A dental implant surgical spacer article, comprising a polyester or polyether polyurethane foam sponge, or combinations thereof, that is autoclavable at 250° F. and has a porosity between not less than 45 pores per inch (ppi) and not more than 105 ppi and configured in size and shape to preserve a volume above a dental implant for coupling an abutment to the dental implant during an osseointegration period.

5. The dental implant surgical spacer article of claim 4, wherein the polyester or polyether polyurethane foam sponge comprises a base end opposite a tapered end.

6. The dental implant surgical spacer article of claim 4, wherein the polyester or polyether polyurethane foam sponge comprises a tapered end to base end weight density ratio of at least 2:1.

7. The dental implant surgical spacer article of claim 4, wherein the polyester or polyether polyurethane foam sponge comprises a conic or truncated conic shape.

8. The dental implant surgical spacer article of claim 4, wherein the polyester or polyether polyurethane foam sponge comprises a pyramid or truncated pyramid shape.

9. An endodontic surgical spacer article, comprising a polyester or polyether polyurethane foam sponge, or combinations thereof, that is autoclavable at 250° F. and has a porosity between not less than 45 pores per inch (ppi) and not more than 105 ppi and that is configured to temporarily preserve a prepared tooth cavity volume until filling material is ready for filling the cavity volume with permanent filling material.

10. The endodontic spacer article of claim 9, wherein the polyester or polyether polyurethane foam sponge comprises an absorbed, adhered or trapped medicinal dosage, or combinations thereof.

11. The endodontic spacer article of claim 9, wherein the polyester or polyether polyurethane foam sponge comprises a base end opposite a tapered end.

12. The endodontic spacer article of claim 9, wherein the polyester or polyether polyurethane foam sponge comprises a tapered end to base end weight density ratio of at least 2:1.

13. The endodontic spacer article of claim 9, wherein the polyester or polyether polyurethane foam sponge comprises a conic or truncated conic shape.

14. The endodontic spacer article of claim 9, wherein the polyester or polyether polyurethane foam sponge comprises a pyramid or truncated pyramid shape.

15. A dental surgical protection article, comprising a polyester or polyether polyurethane foam sponge that is autoclavable at 250° F. and has a porosity between not less than 45 pores per inch (ppi) and not more than 105 ppi and that is configured to protect sensitive or vulnerable mouth tissue from surgical equipment and ambient exposure during an oral surgery.

16. A dental surgical retraction method, comprising placing a polyester or polyether polyurethane foam sponge, as recited at claim 1, at a gingival incision location to retract the gingival flap during an oral surgery.

17. A dental surgical spacer method, comprising placing a polyester or polyether polyurethane foam sponge, as recited at claim 4, in a space next to an embedded dental implant to preserve a spacing for coupling an abutment to the dental implant after an osseointegration period.

18. A dental surgical spacer method, comprising placing a polyester or polyether polyurethane foam sponge, as recited at claim 9, into a prepared tooth cavity volume until filling material is ready for filling the cavity volume with permanent filling material.

19. A dental surgical protection method, comprising placing a polyester or polyether polyurethane foam sponge, as recited at claim 15, against sensitive or vulnerable mouth tissue as protection from surgical equipment impacts and ambient exposure during an oral surgery.

20. A dental article, comprising a polyester or polyether polyurethane foam sponge, or combinations thereof, that is autoclavable at 250° F. and has a porosity between not less than 45 pores per inch (ppi) and not more than 105 ppi, and that is configured for nonadhesively contacting and compressing loose graft material contained therein.

21. The dental article of claim 20, wherein said sponge comprises a 2D or 3D mushroom shape.

22. The dental article of claim 20, wherein said sponge comprises a 2D or 3D football shape.

23. The dental article of claim 20, wherein the polyester or polyether polyurethane foam sponge, or a second polyester or polyether polyurethane foam sponge, is configured for maintaining a volume density integrity of compressed graft material when removing bodily fluids from the bone socket recess by suctioning said fluids through the sponge.

24. The dental article of claim 23, wherein said second sponge comprises a U shape.

25. The dental article of claim 20, further comprising a guided tissue regeneration membrane configured to be disposed over the compressed graft material following said suctioning.

26. The dental article of claim 25, wherein said membrane is configured to remain over said graft material within said socket graft recess during an osseointegration period.

27. A dental bone socket grafting method, comprising:
preparing a bone socket recess defined within a patient's jawbone;
after said preparing said bone socket recess, filling the bone socket recess with loose graft material; and
compressing the loose graft material within the bone socket recess by inserting a nonadhesive polyester or polyether polyurethane foam sponge, or combinations thereof, as recited at claim 20, into contact with the loose graft material therein and applying pressure to said sponge.

28. The dental bone socket grafting method of claim 27, wherein the preparing a bone socket recess comprises shape cutting or drilling into a tooth, or through gum tissue, or into some bone tissue, or combinations thereof.

29. The dental bone socket grafting method of claim 27, wherein the preparing a bone socket recess comprises removing one or more of a decayed tooth, decayed tissue, excess tissue, microbial organic material, or inorganic debris, or combinations thereof.

30. The dental bone socket grafting method of claim 27, comprising suctioning fluid from the bone socket recess through said nonadhesive polyester or polyether polyurethane foam sponge.
